SEQUENCE LISTING (9 sequences)

SEQ ID NO: 1 - DNA, 1989 bp, Artificial Sequence
  Clade C HIV-1 1086.c ENV SOSIP trimer DNA sequence
atggacgctatgaaaagggggctgtgctgtgtgctgctgctgtgcggggctgtgtttgtgtcacccagtcaggaaatccacgccagattccggagaggagctaggatggagggctcgtgggtcacagtttactatggagtgcctgtgtggaaagaagcaaaaactactctattctgtgcatcagatgctaaagcatatgagaaagaagtgcataatgtctgggctacacatgcctgtgtgcccacagatcccaacccacaagaaatggttttggcaaatgtaacagaaaattttaacatgtggaaaaatgatatggtagagcagatgcatgaggatataattagtttgtgggatgaaagcctgaagccatgtgtgaagttgacgccactctgtgtcactttaaattgtacaaatgttaaagggaatgagagtgacaccagtgaagtaatgaaaaattgctctttcaaggcaaccacggaactaaaggataaaaaacataaggtgcatgcgctgttttataaacttgatgtagtaccacttaatggaaacagcagcagctctggagagtatagattaataaattgcaatacctcagccataacacaagcctgtccaaaggtctcttttgacccaattcctttacattactgtgcaccagctggttttgcgattctaaagtgtaataataagacattcaatgggacaggaccatgtcgtaatgtcagcacagtacaatgtacacatggaattaagccagtggtatcaactcaactactgttaaatggtagcctagcagaagaagagataataattagatctgaaaatctgacaaacaatgccaaaacaataatagtacacctcaatgaatctgtaaacattgtgtgtacaagacccaataataatacaagaaaaagtataaggataggaccaggacaaacattctatgcaacaggtgacataataggaaacataagacaggcacattgtaacattaatgaaagtaaatggaacaacactttacaaaaggtaggagaagaattagcaaaacacttccctagtaaaacaataaagtttgaaccatcctcaggaggcgatctagaaattacaacacatagctttaattgtagaggagagttcttctattgcaatacatcagacctgtttaatggtacatacagaaatggtacatacaatcatacaggaagaagttcaaatggaaccatcaccctccaatgcaaaataaaacaaattataaacatgtggcaggaggtaggaagagcaatatatgcgcctcccattgaaggagaaataacatgtaactcaaatatcacaggactactattgctacgtgatggaggtcaatcaaatgaaacaaatgacacagagacattcagacctggaggaggagatatgagggacaattggagaagtgaattatataaatataaagtagtagaaattaaaccattgggagtagcacccactgagtgtaaaagaagagtggtggagagacggagacggagaagagcagtgggaataggagctgtgttccttgggttcttgggagcagccggaagcactatgggcgcagcatcaatgacgctgacggtacaggccaggcaattattgtctggtatagtgcaacagcaaagcaatttgctgagggctccagaggcgcaacagcatatgttgcaactcacggtctggggcattaaacagctccaggcaagagtcctggctatagaaagatacctaaaggatcaacagctcctagggatgtggggctgctctggaaaactcatctgctgtactgctgtgccttggaactccagttggagtaacaaatctcaaaatgaaatttggggcaacatgacctggatgcagtgggacagagaaattaataattacacaaacacaatatataggttacttgaagactcacaaaaccagcaggaaaaaaatgagaaagatttgttagcattggactaa

SEQ ID NO: 2 - protein, 627 aa, Artificial Sequence
  Clade C HIV-1 1086.c ENV SOSIP trimer protein sequence without signal sequence
MEGSWVTVYYGVPVWKEAKTTLFCASDAKAYEKEVHNVWATHACVPTDPNPQEMVLANVTENFNMWKNDMVEQMHEDIISLWDESLKPCVKLTPLCVTLNCTNVKGNESDTSEVMKNCSFKATTELKDKKHKVHALFYKLDVVPLNGNSSSSGEYRLINCNTSAITQACPKVSFDPIPLHYCAPAGFAILKCNNKTFNGTGPCRNVSTVQCTHGIKPVVSTQLLLNGSLAEEEIIIRSENLTNNAKTIIVHLNESVNIVCTRPNNNTRKSIRIGPGQTFYATGDIIGNIRQAHCNINESKWNNTLQKVGEELAKHFPSKTIKFEPSSGGDLEITTHSFNCRGEFFYCNTSDLFNGTYRNGTYNHTGRSSNGTITLQCKIKQIINMWQEVGRAIYAPPIEGEITCNSNITGLLLLRDGGQSNETNDTETFRPGGGDMRDNWRSELYKYKVVEIKPLGVAPTECKRRVVERRRRRRAVGIGAVFLGFLGAAGSTMGAASMTLTVQARQLLSGIVQQQSNLLRAPEAQQHMLQLTVWGIKQLQARVLAIERYLKDQQLLGMWGCSGKLICCTAVPWNSSWSNKSQNEIWGNMTWMQWDREINNYTNTIYRLLEDSQNQQEKNEKDLLALD

SEQ ID NO: 3 - protein, 662 aa, Artificial Sequence
  Clade C HIV-1 1086.c ENV SOSIP trimer protein sequence with signal sequence
MDAMKRGLCCVLLLCGAVFVSPSQEIHARFRRGARMEGSWVTVYYGVPVWKEAKTTLFCASDAKAYEKEVHNVWATHACVPTDPNPQEMVLANVTENFNMWKNDMVEQMHEDIISLWDESLKPCVKLTPLCVTLNCTNVKGNESDTSEVMKNCSFKATTELKDKKHKVHALFYKLDVVPLNGNSSSSGEYRLINCNTSAITQACPKVSFDPIPLHYCAPAGFAILKCNNKTFNGTGPCRNVSTVQCTHGIKPVVSTQLLLNGSLAEEEIIIRSENLTNNAKTIIVHLNESVNIVCTRPNNNTRKSIRIGPGQTFYATGDIIGNIRQAHCNINESKWNNTLQKVGEELAKHFPSKTIKFEPSSGGDLEITTHSFNCRGEFFYCNTSDLFNGTYRNGTYNHTGRSSNGTITLQCKIKQIINMWQEVGRAIYAPPIEGEITCNSNITGLLLLRDGGQSNETNDTETFRPGGGDMRDNWRSELYKYKVVEIKPLGVAPTECKRRVVERRRRRRAVGIGAVFLGFLGAAGSTMGAASMTLTVQARQLLSGIVQQQSNLLRAPEAQQHMLQLTVWGIKQLQARVLAIERYLKDQQLLGMWGCSGKLICCTAVPWNSSWSNKSQNEIWGNMTWMQWDREINNYTNTIYRLLEDSQNQQEKNEKDLLALD

SEQ ID NO: 4 - protein, 6 aa, Artificial Sequence
  BG505 SOSIP trimer protein fragment
DMRGEL

SEQ ID NO: 5 - protein, 7 aa, Artificial Sequence
  BG505 SOSIP trimer protein fragment
QGNRSNN

SEQ ID NO: 6 - protein, 5 aa, Artificial Sequence
  BG505 SOSIP trimer protein fragment
VSKAT

SEQ ID NO: 7 - protein, 4 aa, Artificial Sequence
  BG505 SOSIP trimer protein fragment
WISN

SEQ ID NO: 8 - protein, 5 aa, Artificial Sequence
  BG505 SOSIP trimer protein fragment
SVQGS

SEQ ID NO: 9 - protein, 4 aa, Artificial Sequence
  BG505 SOSIP trimer protein fragment
RNLS

(19) United States
(12) Patent Application Publication
(10) Pub. No.: US 2024/0181040 A1
(43) Pub. Date: Jun. 6, 2024

(54) CLADE C HIV-1 ENVELOPE (ENV) TRIMER IMMUNOGENS, COMPOSITIONS INCLUDING THE CLADE C HIV-1 ENVELOPE (ENV) TRIMER IMMUNOGENS, AND USES THEREOF

(71) Applicant: NOVA SOUTHEASTERN UNIVERSITY, Fort Lauderdale, FL (US)

(72) Inventors: Mark J. Cayabyab, Fort Lauderdale, FL (US); Alexander Bontempo, Fort Lauderdale, FL (US)

(73) Assignees: NOVA SOUTHEASTERN UNIVERSITY, Fort Lauderdale, FL (US); The Forsyth Institute, Cambridge, MA (US)

(21) Appl. No.: 18/284,894

(22) PCT Filed: Mar. 30, 2022
(86) PCT No.: PCT/US2022/022557

§ 371 Date: Sep. 29, 2023

Related U.S. Application Data
(60) Provisional application No. 63/168,077, filed on Mar. 30, 2021.

(51) Int. Cl. A61K 39/21; A61K 39/00; C07K 14/005

(52) U.S. Cl. CPC A61K 39/21; C07K 14/005; A61K 2039/70

(57) ABSTRACT

The invention encompasses a non-naturally occurring clade C human immunodeficiency virus type-1 (HIV-1) 1086.C envelope (ENV) SOSIP trimer protein. This trimer protein contains broadly neutralizing epitopes and epitopes that induce anti-V1/V2 antibodies and thus is an immunogen for creation of HIV-1 vaccines. The invention also includes prophylactic or therapeutic vaccine compositions/kits and methods for using the trimer protein as a component of a vaccine against HIV-1 infection.

### STATEMENT REGARDING FDERALLY SPONSORED RESEARCH OR DEVELOPMENT

This invention was made with government support under DE027249 awarded by the National Institutes of Health. The U.S. government has certain rights to the invention.

### FIELD OF THE INVENTION

The invention generally relates to development of an effective vaccine for prevention of human immunodeficiency virus type-1 (HIV-1) infection and Acquired Immune Deficiency Syndrome (AIDS), particularly to HIV-1 envelope (ENV) glycoprotein trimers that mimic natural envelope glycoproteins of the HIV-1 virion and function as immunogens enabling creation of a vaccine, and most particularly to a clade C HIV-1 1086.C ENV SOSIP trimer immunogen and use (of this immunogen) as a component of an HIV-1 vaccine.

### BACKGROUND

HIV infection and development of AIDS remain major global public health problems with an estimated 1.7 million people newly infected and 690,000 lives lost annually to AIDS-related illnesses, despite the availability of effective treatments (according to the United Nations Programme on HIV/AIDS). Development of an effective HIV vaccine is a top priority to stop this pandemic. The two main vaccine approaches to generate protective immunity being explored are through 1) induction of broadly neutralizing antibodies (bnAbs) and 2) induction of functional antibodies (Abs) to the HIV-1 envelope glycoprotein (ENV) V1-V2 region (V2 apex) with antibody-dependent cellular cytotoxicity (ADCC) and other antiviral functions.

Seven HIV vaccine efficacy trials were conducted in the past three decades and only the 2009 Thai RV144 trial was able to show that an HIV vaccine can induce some level of protection against HIV-1. The 31% efficacy conferred by the RV144 heterologous prime-boost vaccine regimen consisting of canarypox vector ALVAC-HIV [vCP1521] prime followed by AIDSVAX B/E gp120 boost was modest, but it demonstrated that inducing protection through vaccination was feasible (1). Correlates of protection study revealed that the protection was associated with IgG antibody binding to the V2 apex (2). Additionally, in vaccinated participants with low vaccine elicited Env IgA, antibody-dependent cellular cytotoxicity (ADCC), neutralizing antibodies and Env-specific CD4+ T cell responses correlated with protection (2). Subsequent studies to explore possible mechanisms of vaccine-elicited protection show that non-neutralizing antibodies specific to the V2 apex with effector functions such as ADCC, Ab-dependent cellular phagocytosis, and Ab-mediated release of cytokines and complement-mediated killing could potentially play a role in protection against HIV (3-11). Collectively, these findings strongly suggest that inducing anti-V2 apex antibodies with antiviral properties similar to those found in vaccinated participants of the RV144 trial who were protected could be a viable vaccine strategy.

Another promising vaccine approach is through the induction of broadly neutralizing antibodies. To account for HIV-1's tremendous global sequence diversity, a vaccine will most likely have to induce bnAbs (12). Since approximately 20-30% of HIV-infected individuals develop bnAbs, induction of bnAbs through vaccination may be possible (13-15). Moreover, vaccine-elicited bnAbs perhaps could protect against HIV based on the finding that passive transfer of bnAbs in non-human primates conferred protection against SHIV challenge (16-20). In vitro, bnAbs prevent infection of host cells by a broad-spectrum HIV-1 demonstrating that induction of bnAbs will be needed to protect against a tremendous diversity of circulating viruses (21).

Broadly neutralizing antibodies (bnAbs) bind to conserved regions of the native Env trimer spike protein on the virus surface; and therefore, ENV immunogens that structurally resemble the native Env trimer on the virion surface are being explored as vaccines to induce protective bnAb responses. In 2013, the first soluble native-like ENV trimer known as BG505 SOSIP.664 was developed (22, 23). The BG505 SOSIP.664 trimer contains multiple bnAb epitopes and can elicit efficiently autologous tier 2 antibodies in vivo (24, 25). BG505 SOSIP-based trimer vaccines are currently being tested in a phase I human trial (26). Several other native-like SOSIP trimers and other native-like gp140 trimers including uncleaved NFL-, SC-, and UFO-gp140 trimers have been generated (27-40)). Because native-like trimers present multiple bnAb epitopes in ways that best mimic how these epitopes appear on the native Env spike and are capable of eliciting neutralizing antibodies again autologous Tier 2 HIV-1, trimer immunogens are being used in exploratory vaccine strategies that can potentially elicit bnAbs capable of neutralizing a broad spectrum of HIV viruses (25, 41, 42). These innovative vaccine strategies include using multiple trimers as sequential or simultaneous immunogens, targeting the germline bnAb precursors, delivering sequential lineages of trimers derived from infected individuals who developed bnAbs, and presenting trimers as particulate antigens (reviewed in (43)).

In this invention, a novel HIV-1 clade C 1086.c ENV SOSIP trimer as an HIV vaccine immunogen was constructed and its biochemical, structural and antigenic properties were assessed. The clade C 1086.c Env was selected as a trimer immunogen because the 1086.c virus is a neutralization-resistant transmitted founder (T/F) virus and clade C viruses are the most prevalent with over 50% of HIV infections found in sub-Saharan Africa (44, 45). Furthermore, 1086.c ENV was also found to be highly immunogenic (45, 46). Biochemical and structural analyses revealed that the 1086.c ENV SOSIP was a native-like trimer and thermostable. In a comprehensive antigenicity study, the 1086.c SOSIP trimer showed considerable affinity for 16 out of 19 bnAbs assessed. Noteworthy, the 1086.c SOSIP trimer exhibited an overall bnAb binding profile complementary to the BG505 SOSIP.664 trimer. For certain bnAbs however, both trimers shared a similar antigenicity profile.

Interestingly, in comparison to previously reported HIV soluble trimers, the 1086.c SOSIP trimer uniquely displayed potentially protective epitopes in the V2 apex that are recognized with unprecedented affinity by functional V2p antibodies correlated with protection in the RV144 trial (47, 48). The combined binding of the anti-V2 apex PG09 bnAb with the RV144-related CH58 and CH59 mAbs to the 1086.c SOSIP trimer suggests a dynamic V2 apex structure present on the trimer. The preferential binding of bnAbs and V2p antibodies to the 1086.c SOSIP trimer over the non-trimeric 1086.c gp120 and gp140 counterparts underscores the fundamental role played by the trimeric native structure in not only the proper presentation of important bnAb epitopes, as previously shown, but also important V2p epitopes that is now shown in the experiments described herein.

Despite much progress, HIV infection remains a worldwide health crisis, thus development of an effective HIV vaccine is necessary to abating this crisis.

### SUMMARY OF THE INVENTION

The instant invention satisfies this need by providing a novel clade C 1086.c SOSIP trimer vaccine candidate immunogen/antigen that contains broadly neutralizing epitopes as well as potentially protective epitopes in the V2 apex region.

The inability to induce an efficient protective response observed in seven major clinical trials conducted previously are in part due to the sub-optimal immunogenicity of the candidate immunogens. The development of an ENV immunogen that structurally resembles the native Env trimer on the virion surface is considered by many experts to be critical for the induction of protective antibody responses. The ENV protein is a trimer composed of gp41/gp120 heterodimers that are readily recognized by the humoral immune system. The gp41 subunit contains a transmembrane domain in its N-terminal portion and a hydrophobic domain that forms a coiled-coil domain in the trimer responsible for driving membrane fusion and viral entry. The gp120 subunit contains the CD4 and co-receptor binding sites. The non-covalent bond between the transmembrane gp41 and the extracellular subunit gp120 is dependent on membrane anchoring and results in the dissociation of the two subunits in soluble trimers. In 2013, the Moore research group reported a methodology to produce a native-like soluble trimer from an HIV-1 clade A virus isolate (BG505 SOSIP) (49, 25). The soluble native-like trimer was obtained through the introduction of gp41/gp120 intra-subunit disulfide bond and an Isoleucine to Proline mutation in the gp41 coiled coil domain to stabilize the complex in its pre-fusion/native state. In addition to the above stabilizing modifications, the BG505 SOSIP is characterized by both the deletion of the Membrane Proximal Extracellular Region (MPER) to decrease aggregation and the substitution of the wild type Signal peptide with the Tissue Plasminogen activator (tPa) Signal peptide to maximize protein yield during in vitro expression. The BG505 SOSIP trimer showed the ability to induce tier 1, autologous tier 2, and some heterologous tier 2 neutralizing responses in vivo (24). Because of promising preclinical findings, the BG505 SOSIP trimer vaccine is now being tested in a phase I human trial (IAVI W001) by the International AIDS Vaccine Initiative (IAVI).

Along with native trimeric structure, significant Env sequence variability found in circulating HIV strains is an important factor in inducing the protective response. For an ENV vaccine to be protective, induction of broadly neutralizing antibody responses to conserved regions of the trimer is needed. The success of inducing bnAb responses is dependent on maximizing the immunogenicity of conserved bnAb epitopes and minimizing antibody responses to immunodominant non-neutralizing antibody epitopes. Moreover, the geographical distribution of specific HIV clades is an important factor in the design of HIV Env vaccines. Because of tremendous interclade diversity, it is reasonable to believe that Env immunogens should be tailored to target the most common HIV sequences found among a specific geographical region prioritizing those with the best immunogenic profile.

In a general aspect, the invention provides an immunogen or antigen that makes creation of a vaccine against human immunodeficiency virus type-1 (HIV-1) infection possible. A vaccine is contemplated as any substance capable of eliciting an immune response.

In a general aspect, the invention provides a composition for prevention of infection with HIV-1, particularly, but not limited to clade C HIV-1. For example, the composition can elicit an immune response in cells to clade C HIV-1.

In another general aspect, the invention provides an immunogen or antigen that is a modified form of a naturally occurring human immunodeficiency virus type-1 (HIV-1) gene and protein.

In another general aspect, the invention provides compositions and methods for prevention of Acquired Immune Deficiency Syndrome (AIDS).

In another general aspect, the invention provides HIV-1 envelope (ENV) glycoprotein trimers that mimic natural envelope glycoproteins of the HIV-1 virion which function as immunogens enabling creation of a vaccine.

In an embodiment, the invention provides a trimer, protein trimer, or protein trimer fragment that is a non-naturally occurring clade C human immunodeficiency virus type-1 (HIV-1) 1086.c envelope (ENV) trimer having a form stabilized with a disulfide SOSIP mutation and having a mutation that replaces an isoleucine residue at position 559 with a proline residue. By “non-naturally occurring” it is meant that some change, mutation, or alteration has been made to the trimer such that it does not occur naturally or appear in nature. The trimer has been shown to contain broadly neutralizing epitopes and epitopes that induce anti-V1/V2 antibodies. A nucleic acid encoding the trimer has a DNA sequence of SEQ ID. NO:1(FIG. 1). The trimer, without a tPa signal sequence, has an amino acid sequence of SEQ ID NO:2 (FIG. 3A) and the trimer, with a tPa signal sequence, has an amino acid sequence of SEQ ID NO:3 (FIG. 3B).

In another embodiment, the described trimer can be combined with at least one of a pharmaceutically acceptable carrier and an adjuvant to form a pharmaceutical composition.

The phrase “pharmaceutically acceptable carrier” refers to an inactive and non-toxic substance used in association with an active substance, i.e. in this invention the clade C HIV-1 1086.c ENV trimer, especially for aiding in the application/delivery of the active substance. “Inactive”, in this context, refers to inactivity with regard to the activity of the active substance. Non-limiting examples of pharmaceutically acceptable excipients are diluents, fillers, binders, disintegrants, superdisintegrants, flavorings, sweeteners, lubricants, alkalizers/alkalinizing agents, and absorption enhancers/penetration enhancers/permeation enhancers. Pharmaceutically acceptable carriers can have more than one function, a non-limiting e.g. a filler can also be a disintegrant. Additionally, pharmaceutically acceptable carriers may also be referred to as non-medicinal ingredients (NMIs) or pharmaceutically acceptable excipients. Any pharmaceutically acceptable carrier used for production and/or delivery of vaccines is contemplated for use with the inventive trimer.

The term “adjuvant” refers to any substance that enhances or is capable of enhancing an immune response to an antigen or immunogen. Any adjuvant used for production and/or delivery of vaccines is contemplated for use with the inventive trimer.

In an embodiment, an HIV-1 envelope (ENV) trimer from the same clade type and/or from a clade type other than clade C, such as, but not limited to, a clade A BG505 SOSIP trimer, can be combined with the trimer and/or the pharmaceutical composition to form a vaccine cocktail.

In another embodiment, the invention provides an immunogenic composition for eliciting an immune reaction in cells against clade C human immunodeficiency virus type-1 (HIV-1). The composition includes a non-naturally occurring clade C human immunodeficiency virus type-1 (HIV-1) 1086.C envelope (ENV) trimer having a form stabilized with a disulfide SOSIP mutation and having a mutation that replaces an isoleucine residue at position 559 with a proline residue and at least one of an adjuvant and a pharmaceutically acceptable carrier.

In yet another embodiment, the invention provides a prophylactic or therapeutic vaccine against HIV-1, particularly, but not limited to, clade C HIV-1. The prophylactic or therapeutic vaccine is meant to prevent and/or treat HIV-1 and includes the inventive trimer and one or more of a pharmaceutically acceptable carrier, an adjuvant, and an HIV-1 envelope (ENV) trimer from a clade type other than clade C, such as, but not limited to, a clade A BG505 SOSIP trimer. For example, a vaccine according to the invention can include the trimer expressed in nucleic acid (such as, but not limited to DNA, RNA, mRNA) or protein form and be given to a subject in need thereof as an injection. This is an exemplary embodiment only and is not meant to limit the invention. The trimer can be expressed using a recombinant viral vector, such as, but not limited to, an adenovirus vector or a Modified Vaccinia Virus Ankara (MVA). Any method for manufacturing and administering vaccines is contemplated for use with the inventive trimer. An intended subject is a human, but the inventive trimer is contemplated for use by any animal which could benefit from its use.

In another embodiment, the components of the inventive compositions and vaccines can be packaged in containers and assembled in kits together with instructions for use.

In yet another embodiment, the invention provides various methods for use of the inventive trimer. The pharmaceutical compositions, immunogenic compositions, and/or vaccines of the invention can be administered to a subject in need thereof to induce an immune response against clade C human immunodeficiency virus type-1 (HIV-1) infection; to induce production of broadly neutralizing antibodies (bnAbs) against clade C human immunodeficiency virus type-1 (HIV-1) infection; to induce production of antibodies against human immunodeficiency virus type-1 (HIV-1) V2 apex: and to vaccinate a subject against clade C human immunodeficiency virus type-1 (HIV-1) infection.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 shows the modified 1086.c SOSIP DNA sequence designated herein as SEQ ID NO: 1. A tPa signal peptide (shaded sequence) was introduced to enhance expression. Coding for the two cysteines involved in inter-subunit disulfide binding (T605C and A501C) is shown (shaded TGT nucleic acid residue) as well as coding for an isoleucine 559 to proline mutation (shaded CCA nucleic acid residue). The membrane-proximal external region (MPER) was deleted to reduce aggregation and enhance solubility.

FIG. 2 shows the VRC8400-1086.cSOSIP plasmid map. The 1086.c SOSIP insert, location of tPa signal, furin cutting site, and SOSIP specific mutation are shown.

FIGS. 3A-C show the 1086.c SOSIP trimer protein sequence and design scheme.

FIG. 3A shows alignment between the 1086.c SOSIP protein (designated herein as SEQ ID NO:2, without the tPa signal sequence) and the 1086.c gp160 protein. The introduction of the enhanced furin cutting site (RRRRRR, amino acid residues 504-509 of SEQ ID NO:2) is shown as well as the cysteines involved in inter-subunit disulfide binding and the isoleucine 559 to proline mutation.

FIG. 3B shows alignment between 1086.c SOSIP protein (designated herein as SEQ ID NO:3, with the tPa signal sequence) and the BG505 SOSIP 664 protein (protein fragments shown as SEQ ID NOS:4-9). The tPa signal sequence (amino acid residues 1-35 of SEQ ID NO:3), differences in N-glycosylation sites, and important glycosylation sites specific to potent neutralizing antibody epitopes (such as PGT121, PGT128, and 2G12) are shown. Additionally, the position of variable loops 1, 2, and 3 as well as the furin cutting site (RRRRRR, amino acid residues 504-509 of SEQ ID NO.3) are shown.

FIG. 3C is a schematic illustration of the SOSIP design. The SOSIP design is characterized by introduction of a disulfide bond between gp120 (A501C) and gp41 (T605C) subunits of HIV-1 envelope glycoprotein. The 1559P mutation enables stabilization of the trimer in its native configuration preventing reorganization of the gp41 subunit upon CD4 binding. Gp41Ecto=gp41 ectodomain (gp41 AMPER).

FIGS. 4A-D show data from biochemical and structural analysis of the 1086.c SOSIP trimer protein.

FIG. 4A shows an elution profile of 1086.C SOSIP trimer protein after SEC/DEAE chromatography (left panel). The trimer peak is enclosed in the grey shaded box. Elution volume obtained was ˜56 ml with a chromatography flow of 0.7 ml/min. After purification, analytical SEC shows a single peak elution profile demonstrating high trimer purity (right panel).

FIG. 4B shows a Blue Native PAGE Analysis in which migration of 1086.c SOSIP (lane 2), the BG505 SOSIP (lane 3), and NativeMark protein ladder (lanel) are shown. 2 μg of trimer protein were loaded per well. The 1086.c SOSIP trimer (indicated with an arrow) had a MW of roughly 700 KDa.

FIG. 4C shows Negative Stain Electron Microscopy in which purified 1086.c SOSIP trimer was compared with BG505 SOSIP trimer by NS-EM using a Tecnai G² Spirit BioTWIN at 80 KV. Scale bar corresponds to 50 nm in width.

FIG. 4D shows an analysis of 2D averages of the 1086.c SOSIP trimer: 5680 single trimer particles were automatically picked with crYOLO and clustered in 120 average stacks using ISAC software. In the lower left corner of each average the size of the stack is indicated. Both the trimer and non-trimer NS-EM images and averages are shown.

FIG. 5 shows a nanoSDF thermal stability analysis of 1086.c SOSIP trimer protein. Thermal stability of the 1086.c SOSIP trimer compared to the BG505 SOSIP.664 trimer was assessed using nanoSDF. The upper panel shows the 350/350 nm fluorescence ratio (y-axis) versus temperature (x-axis). The first derivative of the nanoSDF curve is shown in the lower panel. The melting temperatures of the 1086.c SOSIP and BG505 SOSIP.664 trimers were measured at 58.2° C. and 63.01° C., respectively.

FIG. 6 shows an antigenicity study of 1086.c SOSIP trimer. Twenty-two anti-HIV-1 envelope glycoprotein antibodies were tested for assessing the antigenicity of 1086.c SOSIP trimer compared to BG505 SOSIP.664 trimer construct. The binding kinetic constants Kon, Koff and KD (affinity) are shown with respect to the binding of the trimers to: V2 Apex-specific bnAbs (PG09, PGT16, PGT145, and 697-30D); V2 apex-specific non-neutralizing V2p mAb CH58; CD4 binding site (CD4BS)-specific bnAbs (N6, 3BNC117, HJ16, b12, CH106, VRC01 and VRC03); V3/glycan patch-specific bnAbs (2G12, 10-1074, 3869, PGT121, PGT126, PGT128 and 447-52); and gp41-gp120 interface-specific bnAb (35022). The binding activity of the non-neutralizing antibodies, F105 and 39F to the trimers are also shown. No binding (NB) and poor binding (PB) pairs are indicated.

FIGS. 7A-B show a comparative V1-V2 loop antigenicity of the 1086.c SOSIP trimer, uncleaved 1086.c gp140, and monomeric 1086.c gp120.

FIG. 7A shows the binding kinetic constants Kon, Koff and KD (affinity) of V2p (HG107, HG12, CH58 and CH59), V2i (697-30D), and V2q (PG09 and PG16) and V2qt (PGT145) specific antibodies to 1086.c SOSIP trimer, uncleaved 1086.c gp140 and monomeric 1086.c gp120. No binding pair (NB) is indicated.

FIG. 7B shows a BLI analysis of CH58, CH59 and PG09 binding to 1086.c SOSIP trimer, uncleaved 1086.c gp140 and monomeric 1086.c gp120. The sensograms show the binding shift (nm) over time (seconds).

FIG. 8 shows neutralization sensitivity of BG505 T332N and 1086.c pseudo-typed viruses. Neutralization sensitivity (IC50) of the pseudo-typed viruses to a panel of 27 broadly neutralizing antibodies (bnAbs) and non-neutralizing anti-HIV-1 antibodies are shown.

FIG. 9 is a flow chart showing the steps of the protocol for expression of the 1086.c SOSIP trimer protein in Modified Vaccinia Virus Ankara (MVA).

FIGS. 10A-C show data from expression of the 1086.c SOSIP trimer protein in MVA.

FIG. 10A is a micrograph evidencing production of rMVA expressing 1086c SOSIP trimer protein. A DF-1 cell colony infected with a GFP+ rMVA is shown. Using fluorescence microscopy, DF-1 cells with fluorescence indicate successful production and infection with rMVA expressing 1086c Env and GFP.

FIG. 10B shows purification of the 1086.C SOSIP trimer protein produced by rMVA. The figure is a SEC elution chromatogram of GNL purified protein from DF-1-rMVA 1086c SOSSIP infected cells supernatant. The chromatogram showed a single peak with a retention volume of around 51 ml.

FIG. 10C shows expression of the 1086.C SOSIP trimer protein produced by rMVA. BN-PAGE Coomassie of the peak (FIG. 10B) obtained from SEC is compared with 1086c SOSIP and bg505 SOSIP. The rMVA derived protein is compatible with protein aggregates.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described immunogens, vaccines, and/or methods along with any further application of the principles of the invention as described herein, are contemplated as would normally occur to one skilled in the art to which the invention relates.

Development of an effective HIV vaccine remains a public health priority. An HIV vaccine that can elicit broadly neutralizing antibodies (bnAbs) or functional antibodies that bind the HIV envelope glycoprotein variable regions 1 and 2 (V2 apex) will likely be protective against HIV. A novel HIV-1 transmitted-founder (T/F) clade C 1086.c ENV SOSIP trimer was generated in the experiments described herein that was recognized by many bnAbs specific to conserved regions of V2 apex, CD4 binding site, and V3/glycan patch. Unlike any other trimers, the 1086.c SOSIP trimer appears to have a dynamic V2 apex structure containing both bnAb epitopes and potentially protective epitopes recognized by monoclonal antibodies isolated from RV144 trial vaccinees.

### Experimental Procedures

### EXAMPLE 1

### Materials and Methods

Antibodies and HIV ENV proteins: The HIV-1 Env 1086.C D7gp120 and 1086.C gp140C envelope glycoproteins, CD4-IgG2, anti-HIV-1 envelope monoclonal antibodies 2G12, PGT121, PGT126, PGT128, 39F, 447-52D, F105, 3869, 35O22, CH58, CH59, PG09, PG16, PGT145, IgG1 b12, HG107, HG120, 697-30D, 3BNC117, N6, VRC-CH31, HJ16, VRC01 and VRCO3 were obtained from the NIH AIDS Reagents Program (Germantown. MD). The BG505 SOSIP.664 envelope trimer protein, pcDNA3.1-Furin and pPPI4-BG505 T332N gp160 plasmids were kindly provided by Dr. John P. Moore, Cornell University, Ithaca, NY. The VRC8400 Expression plasmid was kindly donated by Dr. Gary Nabel (NIH).
ENV construction, expression and purification: The 1086.c ΔMPER tPa Signal peptide gene fragment was genetically synthetized (Invitrogen. Carlsbad. Ca). The synthetic gene was cloned in the multiple cloning site of VRC8400 expression plasmid. After cloning we performed mutagenesis (QuickChange multi-site-Directed mutagenesis kit, Agilent technology, Santa Clara. CA.) to insert the Cysteines required for the formation of the disulfide bound (A501C and T605C), the 1559P and the enhanced Furin cutting site RRRRRR (for example, amino acid residues 504-509 of SEQ ID NO:2). Env proteins were produced using Expi293 expression system (Gibco, Carlsbad, Ca.) according to manufacturer's protocol. Briefly, the day before transfection, 800 ml of culture at 2.7×10⁶ cells/ml was prepared from a 3-5×10⁶ cells/ml confluent culture. On the day of transfection, culture was diluted to a final concentration of 3×10⁶ cells/ml in a volume of 800 ml. 800 μg 1086.c-VRC8400 and 266 μg Furin-pcDNA3.1 of plasmids were added to 12 ml of Opti-Mem reduced medium (Gibco, Carlsbad, CA). 640 μl of ExpiFectamine293 reagent (Gibco, Carlsbad, Ca.) was diluted in 11.2 ml of Opti-Mem reduced medium and incubated for 5 minutes at RT. After incubation, the ExpiFectamine293 solution was slowly added to the DNA dilution and incubated for 15 minutes at RT. After incubation, the solution was added to the Expi293 culture dropwise. The transfected cell culture was grown in a 37° C. and 8% CO₂ incubator for 7 days in a 3-liter plane bottom PETG Erlenmeyer bottles (Nalgene, Carlsbad, Ca.) with continuous shaking at 125 RPM. Following incubation, transfected cells were centrifuged at 3000 g for 30 minutes and supernatant was clarified by 0.22 μm iltration. Subsequently, the supernatant was processed through a Galanthus Nivalis Lectin Colum (GNL) (Vector laboratories, Burlingame, CA) by gravity. The column was washed with 2 volumes of washing buffer (0.5M NaCl in PBS) and then rinsed with PBS. After closing the column stopcock, 2 volumes of Methyl α-D-mannopyranoside 1M (Sigma Aldrich, St. Louis, MO) in PBS/pH7.4 were added to the column and incubated for 1 minute. The eluted sample was collected in a storage bottle filled with 2 volumes of TN-75 (TRIS 20 nM; NaCl 75 mM; pH8.0) and subsequently concentrated to a final volume of 1 ml using Amicon® Ultra-15 100 KDa Centrifugal Filter Units (Millipore, Burlinghton, MA).
Tandem SEC/anion exchange chromatography: The AKTA 100 Explore FPLC was equipped with a HighLoad 16/600 Superdex 200 pg Column (GE healthcare, Chicago, IL) and two HiTrap DEAE Sepahrose columns (GE Lifescience, Chicago, IL) mounted in tandem. The FPLC system was equilibrated with TN-75 (TRIS 20 nM; NaCl 75 mMl pH8.0) until stabilization of 215 nm and 280 nm absorbance signal was obtained. The protein concentrated from GNL chromatography was injected and processed with a flow rate of 0.7 ml/min and 2-5 ml fractions were collected. The trimer-specific fraction was then pooled and concentrated to a final 500 ul volume using Amicon® Ultra-15 100 KDa Centrifugal Filter Units (Millipore, Burlinghton, MA). The final protein sample was quantified by 280 nm absorbance using a nanodrop 2000 (ThermoFisher, Waltham, MA).
BN-PAGE analysis: The HIV envelope proteins (2 μg) were resuspended in Native PAGE sample buffer and loaded into Native PAGE 4-16% Bis-Tris gel (Invitrogen, Carlsbad, CA). Electrophoresis was conducted at 120 V for 2 hours in a light blue solution containing Native PAGE running buffer and Native PAGE cathode additive (Invitrogen, Carlsbad, CA). The HIV proteins were then stained with GelCode Blue Safe Protein Stain (Thermo Scientific, Rockford, IL, USA) for 1-1.5 hours and analyzed using the Innotech FluorchemQ image III system. NativeMark Protein Standard (Invitrogen, Carlsbad, CA) was used as a molecular weight marker.
NS-EM analysis and 2D classification: Negative Staining Electron microscopy (NS-EM) Analysis. NS-EM was performed at the Harvard Medical School Electron Microscopy Facility (Boston, MA). Carbon coated meshes were glow discharged at 25 mA for 20 seconds. The meshes were floated over with 5 ul of sample for 20 seconds. After drying with filter paper, the meshes were washed for 30 seconds, dried and stained with 7.5% m/v uranyl formate. Images were captured on a Tecnai G² Spirit BioTWIN microscope with 68.000× direct magnification at 80 kV using Homamatsu ORCA HR camera. For 2D classification, a total of 5680 NS-EM particles projection were automatically picked using crYOLO and analyzed for 2D averages by ISAC software. crYOLO and ISAC are part of the SParx and High Resolution Electron Microscopy suite (SPHIRE).
Nano Differential Scanning Fluorimetry (nanoDSF): ENV protein at 0.25 mg/ml 20 ul was loaded into nanoDSF Grade Standard Capillary (NanoTemper, Munich, Germany). Thermal ramp ranged from 25 to 95° C. with an incremental rate of 1° C. per minute. Data were collected using a Prometheus NT. Plex (NanoTemper, Munich, Germany) at Harvard Medical school's Macromolecular Interaction Center and analyzed with PR.Stability Analysis software (NanoTemper, Munich, Germany).
Bio-Layer Interferometry (BLI) Analysis: BLI was performed at the Harvard University Center for Macromolecular Interactions (Boston, MA). Anti-Human Fc capture biosensors (Fortebio, Fremont, CA) were loaded with 10 μg/ml antibody solution for 2 minutes. Biosensors underwent a cycle of binding and dissociation as follows: basal signal for 2 minutes, binding step for 5 minutes and dissociation for 10 minutes. ENV proteins diluted in running buffer (0.1% bovine serum albumin in PBS pH7.4) ranging from 3.9 nM to 250 nM were assessed for binding. Data were processed using Fortebio software suite applying a global fit model. Binding measurement was obtained using a Fortebio Octet Red 384 system. Binding on-rate constant, Kₒₙ was expressed as

$$\frac{1}{M * s}$$

and off-rate constant, K_{off} as

$$\frac{1}{s} .$$

The affinity constant, K_{D} was calculated using the kinetic equation:

$${K}_{D} = \frac{{K}_{off}}{{K}_{on}}$$

and expressed in molarity.
Pseudovirus production: Pseudo lentivirus was produced using a three-plasmid system and transfection of 293T cells (ATCC, Manassas, VA) following Addgene Lentivirus Production protocol. In brief, the day before transfection, 10 cm petri dishes containing 3.8×10⁶ 293T cells were grown overnight at 37° in a 5% CO2 incubator. Cloroquine diphosphate was added to 25 uM final concentration and incubated for 5 hours. A cell solution containing 70.77 ug linear Polyethylenimine MW 25000 KDa (PEI) (Polysciences, Warrington, PA) in 500 ul Opti-MEM reduced medium was prepared. A second cell solution containing 10.74 μg pCMV-dR8.2 plasmid (Addgene, Watertown, MA), 9.7 μg pALPS tat-P2A-rev plasmid (Cat. #101331 Addgene, Watertown, MA) and 3.09 μg of VRC8400 1086.c gp160 wt or pcDNA3.1 BG505 T332N gp160 in 500 μl Opti-MEM reduced medium was prepared. The PEI solution was added to the DNA solution dropwise and incubated RT for 15-20 minutes. The final solution was then added to the cells culture dropwise while gently shaking. The day after, the medium was replaced with fresh DMEM (GIBCO, Carlsbad, CA) containing 10% FBS and 1% PEN/STREP. Supernatant containing virus was collected after 3-4 day of cell incubation and clarified through 0.4 μm filtration.
Neutralization assay: The lentivirus suspension was seeded in 96-well plate at 100 ul per well in combination with 50 ul six-serial dilution of each antibody to be tested. The mixture was incubated at 37° C. and 5% CO2 while preparing a suspension of 1×105 TZM-bl cells in complete DMEM. DEAE was added to the suspension for a final concentration of 50 ug/ml. Each virus/Ab containing well was added with 100 ul of cell suspension (1×104 cells×well) and incubated for 3 days. After incubation. 150 ul medium per well was removed and replaced with 100 ul BriteLite Plus (PerkinElmer, Waltham, MA). Cells were resuspended by pipetting 3-5 times and incubated for 1 minute. 150 ul of cell suspension was then transferred to 96-well black plates and luminescence was detected by SpectraMax iD5 (Molecular Devices, San Jose, CA). IC50 was estimated by logistic curve fit.

### RESULTS

### Expression of the 1086.c SOSIP Trimer with Biochemical and Structural Analysis

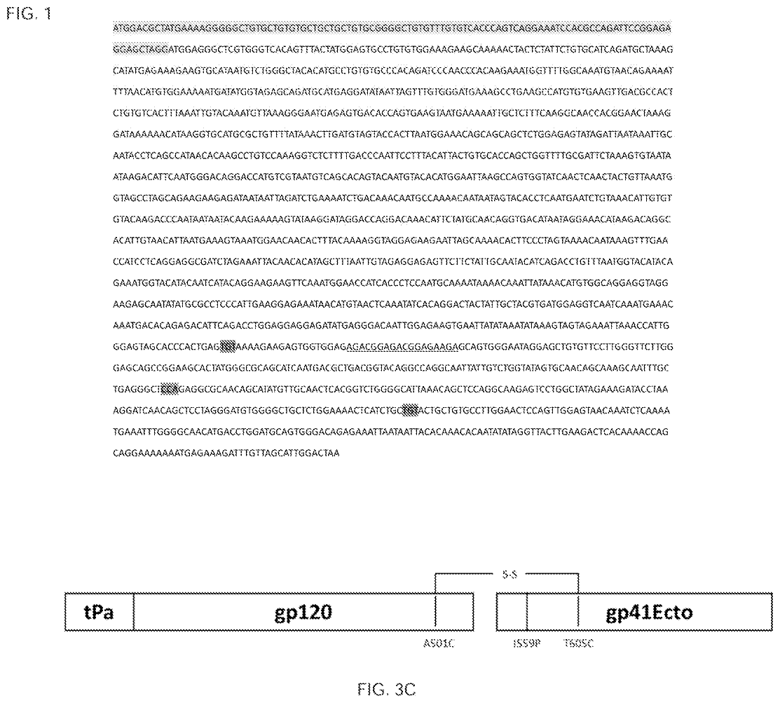

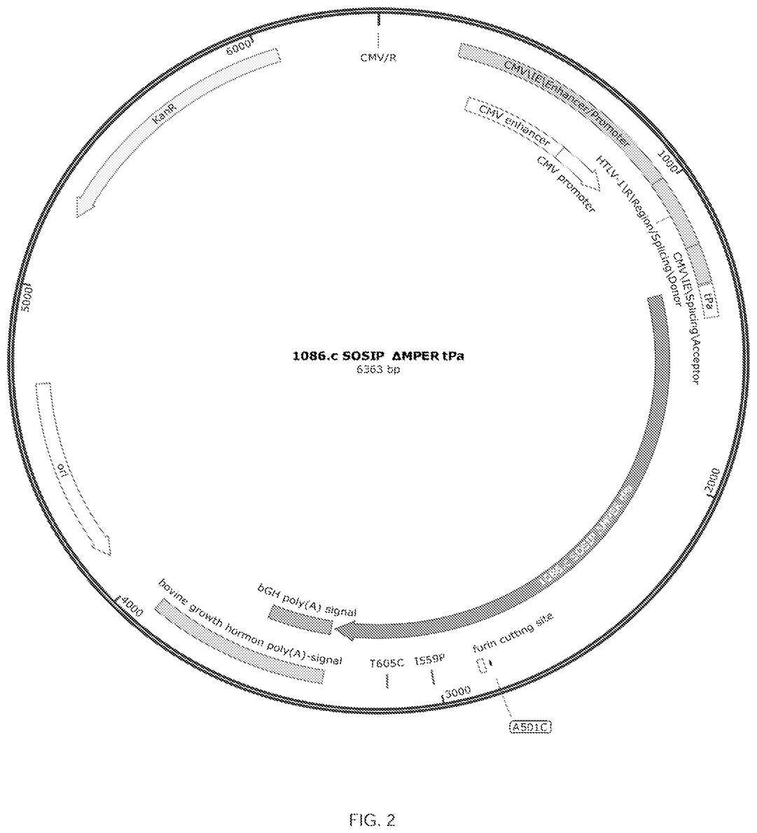

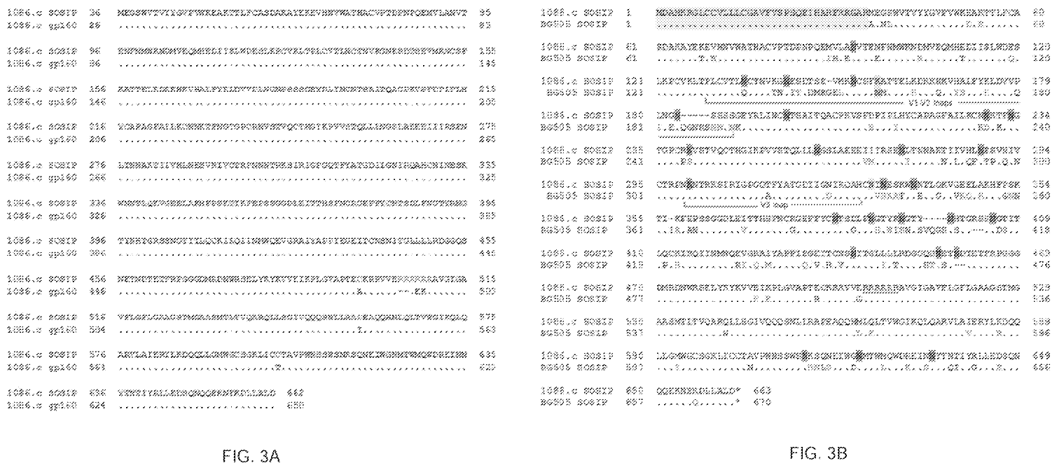

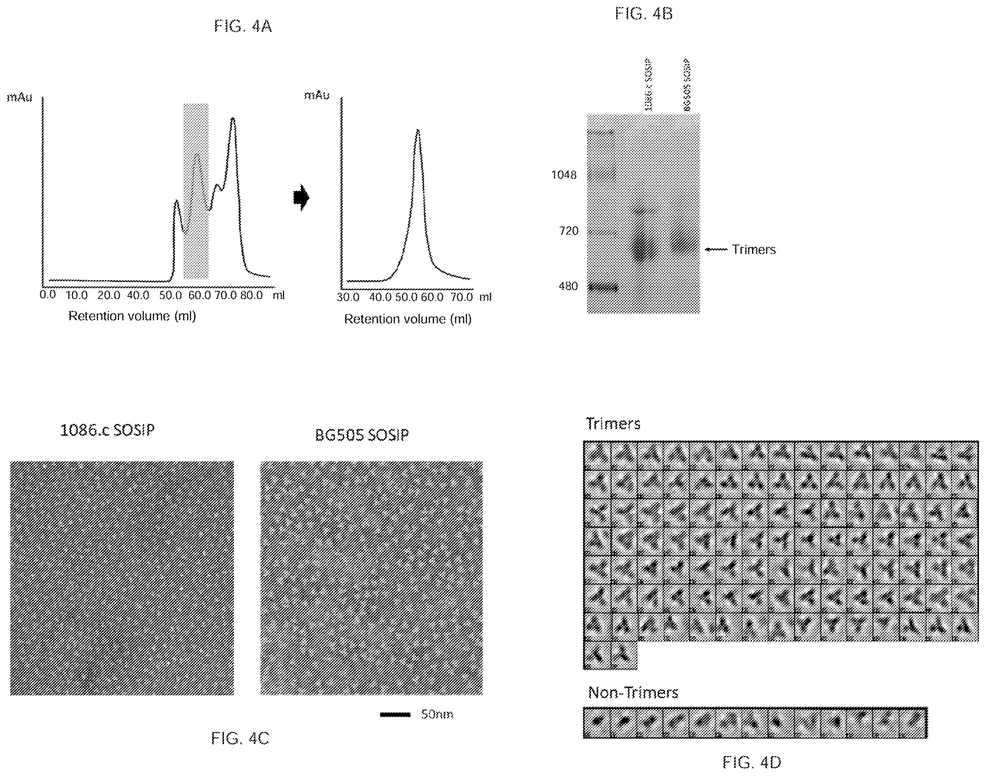

A novel SOSIP gp140 trimer based on the 1086.c Env glycoprotein was produced and characterized in this study (FIGS. 1, 2 and 3A-C). HIV-1 1086.c. is a clade C transmitted/founder (T/F) virus isolated from an acutely infected HIV-1 patient (45). Using a SOSIP design. the soluble native-like trimer was generated through introduction of a gp41/gp120 intra-subunit disulfide bond (SOS) and an Isoleucine to Proline mutation (IP) in the gp41 coiled coil domain to stabilize the complex in its pre-fusion/native state (FIG. 3B) (25). In addition to the above stabilizing modifications, the furin cutting site RRRRRR sequence (amino acid residues 504-509 of SEQ ID NO.3) was introduced to enhance gp120/gp140 cleavage and the Membrane Proximal Extracellular Region (MPER) was deleted to decrease aggregation (FIG. 3B) (25, 49). The 1086.c SOSIP trimer was expressed successfully in Expi293 cells transiently co-transfected with the VRC8400 expression plasmid containing the tPA-ΔMPER 1086.c SOSIP gene fragment and the Furin gene (FIG. 3B). The trimer was purified by a dual step chromatographic procedure using GNL and SEC/DEAE tandem chromatography. SEC/DEAE chromatography elution profile showed four partially overlapping peaks compared to the three-peak profile observed during BG505 SOSIP.664 purification (FIG. 4A, left panel) (25). Even though the SEC/DEAE chromatography was performed, a residual peak containing protein aggregates was observed. The trimer peak fraction was collected, and SEC chromatography of the trimer solution showed a single well-defined peak at 56.5 ml indicating a homogenous sample (FIG. 4A, right panel). The trimer protein yield was approximately 1.0 mg per 1 L of transfected supernatants. BN-PAGE analysis of the 1086.c SOSIP trimer revealed a molecular weight of approximately 700 kDa (FIG. 4B).

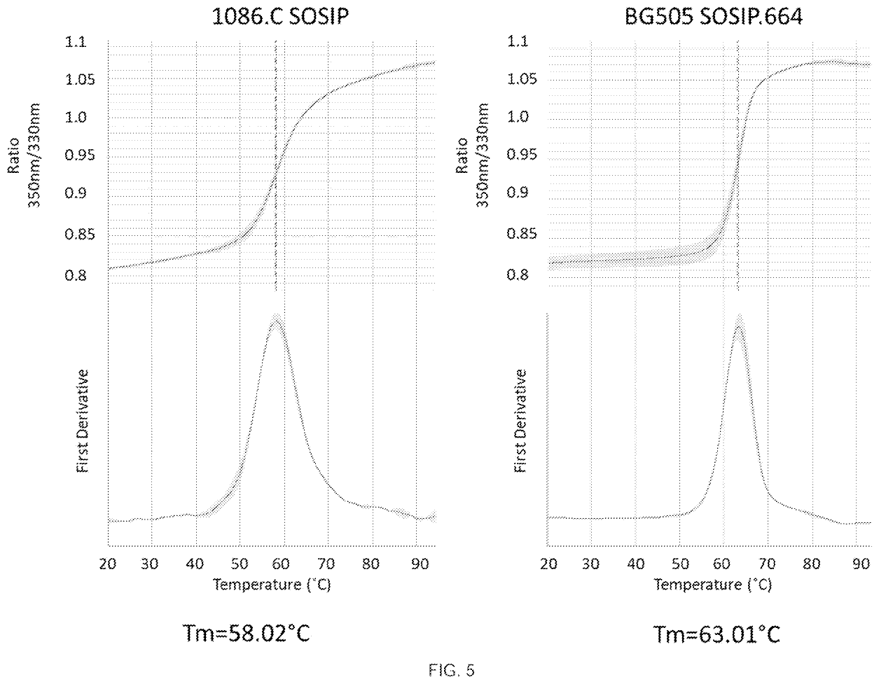

Structural analysis by NS-EM showed that the purified 1086.c SOSIP trimer sample contained well-dispersed and well-ordered native-like trimeric particles, similar to BG505 SOSIP.664 (FIG. 4C). The 2D average analysis of the 1086.C SOSIP trimer in solution revealed the presence of 108 homogeneous trimeric-like averages and 12 averages with non-trimeric structure (FIG. 4D). Out of the 5,680 particles analyzed 5,164 were considered trimers in structure, indicating trimer purity of approximately 91%. Using nanoSDF thermostability assay to measure stability. it was found the 1086.c SOSIP trimer has a melting temperature of 58.02° C. (FIG. 5). In contrast, BG505 SOSIP trimer showed a fast denaturation step with a melting temperature of 63.01° C. (FIG. 5). Both trimer proteins are considered highly stable with a single-step denaturation profile (FIG. 5).

### Antigenicity of 1086.c SOSIP Trimer

The antigenicity of the 1086.c SOSIP trimer was next assessed by conducting binding assessments of bnAbs to the trimer. The bnAb binding profile of the 1086.c SOSIP trimer was compared with the BG505 SOSIP.664 using an extensive panel of 19 bnAbs including antibodies recognizing well-characterized immunogenic portions of the ENV protein such as V2 apex, CD4 binding site (CD4 bs), V3 loop/glycan patch, and the gp120/gp41 interface. The affinity of the RV144-related functional CH58 mAb to both SOSIP trimers was also compared.

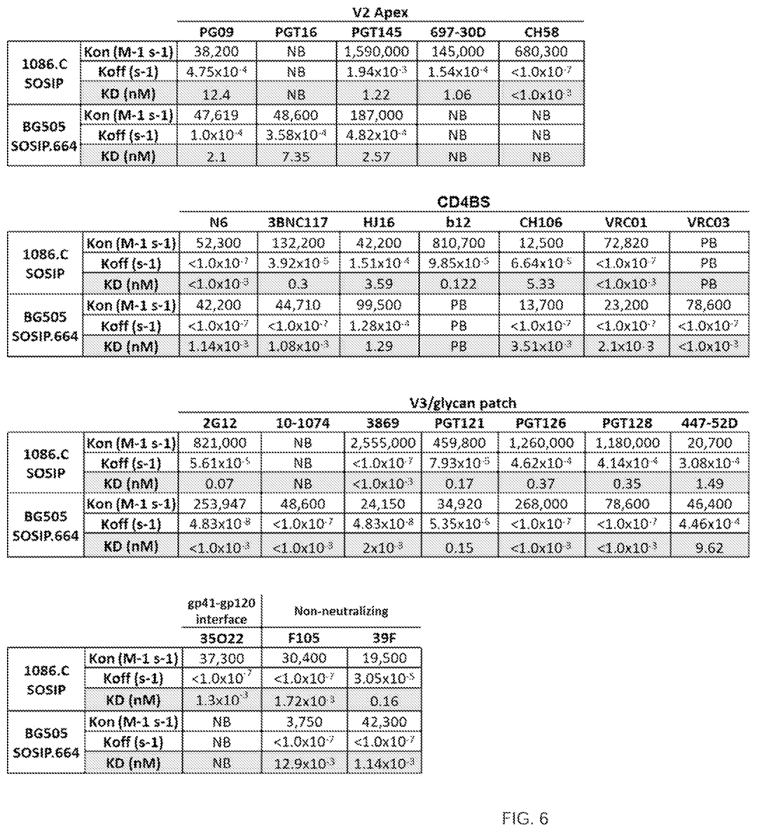

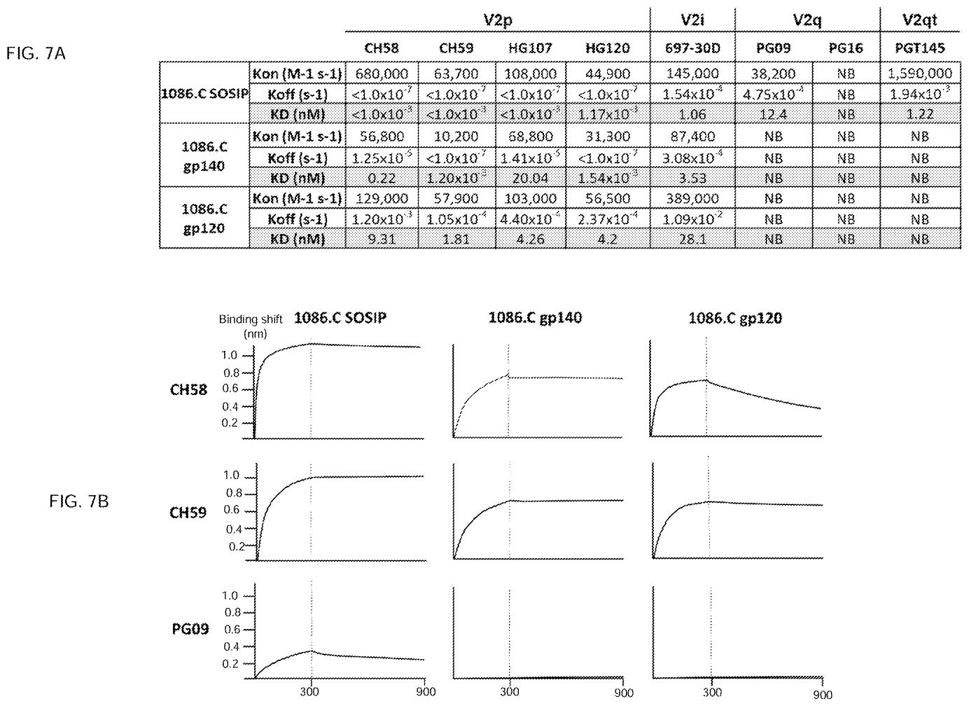

The five V2 apex-specific bnAbs (PG09, PG16, PGT145 and 697-30D) and RV144-related functional CH58 mAb (48, 50-52) were analyzed. PG09 showed strong affinity to both trimers with the bnAb binding the BG505 SOSIP.664 trimer stronger (2.1 nM K_{D})) than the 1086.c SOSIP trimer (12.4 nM K_{D}) (FIG. 6). PG16 only interacted with the BG505 SOSIP.664 trimer (7.35 nM K_{D}) (FIG. 7A). The trimeric-specific PGT145 bound to both Env trimers, with higher affinity to the 1086.c SOSIP trimer (1.22 nM K_{D}) than BG505 SOSIP.664 trimer (2.57 nM K_{D}) (FIG. 6). The 697-30D antibody showed no affinity to BG505 while it bound significantly to the 1086.c trimer (1.06 nM K_{D}) (FIG. 6). The RV144 protection-correlated CH58 mAb only bound with strong affinity to the 1086.c SOSIP trimer (<1×10⁻³ nM K_{D}) but not to the BG505 SOSIP.664 trimer (FIG. 6).

The antigenicity of the trimers to CD4bs-specific 3BNC117, HJ16, CH106, VRC01, VRC03, b12 bnAbs and CD4-Ig was assessed and the most potent bnAb discovered so far. N6 (53-59). Most of the CD4bs-specific bnAbs recognized both 1086.c SOSIP and BG505 SOSIP.664 trimers. However, the BG505 SOSIP.664 was recognized more strongly than the 1086.c SOSIP trimer to 3BNC117 (BG505 SOSIP.664 trimer=1.08×10⁻³ nM K_{D} vs. 1086.c SOSIP trimer=0.30 nM K_{D}). HJ16 (BG505 SOSIP.664 trimer=1.29 nM K_{D} vs. 1086.c SOSIP trimer=3.59 nM K_{D}). CH106 (BG505 SOSIP.664 trimer=3.51×10⁻³ nM K_{D} vs. 1086.c SOSIP trimer=5.33 nM K_{D}) and VRC03 (BG505 SOSIP.664 trimer<1.03×10⁻³ nM K_{D} vs. 1086.c SOSIP trimer=poor binding) (FIG. 6). In contrast, the 1086.c SOSIP trimer outperformed BG505 SOSIP.664 with respect to N6 binding (1086.c SOSIP trimer <1.0×10⁻³ nM K_{D} vs. BG505 SOSIP.664 trimer=1.14×10⁻³ nM K_{D}), VRC01 binding (1086.c SOSIP trimer <1.0×10⁻³ nM K_{D} vs. BG505 SOSIP.664 trimer=2.1×10⁻³ nM K_{D}) and b12 binding (1086.c SOSIP trimer=0.12 nM K_{D} vs. BG505 SOSIP.664 trimer=poor binding) (FIG. 6).

With respect to the V3/glycan patch antigenicity. the binding of 2G12, PGT121, 10-074, 3869, PGT126, PGT128, and 447-52D to the trimers (51, 60-64) was assessed. Binding affinity to PGT121 was similar for both trimers. bnAbs that bound to BG505 SOSIP.664 with higher affinity than to 1086.c SOSIP trimer include 10-1074 (BG505 SOSIP.664 trimer<1.0×10⁻³ nM K_{D} vs. 1086.c SOSIP trimer=no binding). PGT126 (BG505 SOSIP.664 trimer<1.0×10⁻³ nM K_{D} vs. 1086.c SOSIP trimer=0.37 nM K_{D}), PGT 128 (BG505 SOSIP.664 trimer<1.0×10⁻³ nM K_{D} vs. 1086.c SOSIP trimer=0.35 nM K_{D}), and 2G12 (BG505 SOSIP.664 trimer<1.0×10⁻³ nM K_{D} vs. 1086.c SOSIP trimer=0.07 nM K_{D}) (FIG. 6). In contrast, the 1086.c trimer was recognized with stronger affinity than the BG505 SOSIP.664 trimer by 3869 (1086.c SOSIP trimer<1.0×10⁻³ nM K_{D} vs. BG505 SOSIP.664 trimer=2×10⁻³ nM K_{D}) and 447-52D (1086.c SOSIP trimer=1.49 nM K_{D} vs. BG505 SOSIP.664 trimer=9.62 nM K_{D}) (FIGS. 4A-D). With respect to PGT121, this bnAb bound both trimers with similar affinity (1086.c SOSIP trimer=0.17 nM K_{D} vs. BG505 SOSIP.664 trimer=0.15 nM K_{D}) (FIG. 6).

The binding of the gp41-gp120 interface specific antibody 35022 was also assessed and this antibody only had affinity to the BG505 SOSIP.664 trimer (1.3×10⁻³ nM K_{D}) (FIG. 6) (65). Lastly, the following non-neutralizing antibodies bound to both 1086.c SOSIP trimer and as previously shown to the BG505 SOSIP.664 trimer: CD4bs-specific antibody, F105 (1086.c SOSIP trimer=1.72×10⁻³ nM K_{D} vs. BG505 SOSIP.664 trimer=12.9×10⁻³ nM) and V3-specific antibody, 39F (1086.c SOSIP trimer=0.16 nM K_{D} vs. BG505 SOSIP.664 trimer=1.14×10⁻³ nM K_{D} vs.) (FIG. 6) (25, 40).

### Binding of Anti-V2 Apex bnAbs and RV144 Functional Antibodies to the 1086.c SOSIP Trimer, 1086.c gp140 and 1086.c gp120 Monomer

The comparative antigenicity study between BG505 SOSIP.664 trimer and 1086.c SOSIP trimer showed that the V2 apex of 1086.c trimer is unique among previously reported trimer constructs in that it was recognized by not only RV144-related antibodies that were previously shown to recognize contained linear epitope, but also by bnAbs that were shown to bind to quaternary epitopes (FIG. 6). A more comprehensive assessment of 1086.c SOSIP V2 apex antigenicity was performed by using various anti-V2 apex bNAbs and RV144-related antibodies. Whether the trimeric structure of the 1086.c trimer has an effect on the ability of anti-V2 apex bnAbs and functional RV144 antibodies that recognize linear epitopes by determining the antigenicity profile of our 1086.c SOSIP trimer compared to the monomeric gp120 and non-trimeric uncleaved gp140 form of the 1086.c Env was assessed (66-68). This comparative binding analysis underscores the influence of the Env structure over the affinity of bnAb and functional antibodies belonging to V2p, V2i, V2q and V2qt class of antibodies that recognize the V2 apex region of HIV-1 Env glycoprotein (66).

The V2p family of antibodies recognizes the a-helix in the V2 C-strand and include non-neutralizing anti-V2 apex antibodies with antiviral properties that are suggested to be important for protection based on post-hoc analysis of the RV144 (66). Binding to the 1086.c SOSIP trimer by anti-V2 apex antibodies CH58, CH59, HG107 and HG120 that were isolated from RV144 vaccinated individuals and are classified as V2p antibodies that recognize a linear epitope was assessed (48). All the three 1086.c Env glycoprotein forms were recognized by CH58, with the strongest binding to the 1086.c SOSIP trimer (<1.03×10⁻³ nM K_{D}) followed by the gp140 (0.22 nM K_{D}) and gp120 (9.31 nM K_{D}) (FIGS. 7A-B). For CH59, binding to the 1086.c SOSIP trimer (<1.0×10⁻³ nM K_{D}) was the strongest followed by gp140 (1.20×10⁻³ nM K_{D}) and gp120 (1.81 nM K_{D}) (FIGS. 7A-B). The affinity for HG107 was highest to the 1086.c SOSIP trimer (1.17×10⁻³ nM K_{D}), followed by the gp140 (4.2 nM K_{D}) and the gp120 (20.04 nM K_{D}) (FIG. 7A). Binding of HG120 was strongest to the 1086.c SOSIP trimer (<1.0×10⁻³ nM K_{D}). then to the gp140 (1.54×10⁻³ nM K_{D}), and gp120 (4.2 nM K_{D}) (FIG. 7A).

The V2i antibody family is comprised of 7 members that recognize a discontinuous epitope found on the α4β7 integrin binding site (52, 66, 69). The antigenicity study with the V2i antibody 697-30D showed that the V2i bound strongest to the 1086.c SOSIP trimer (1.06 nM K_{D}) followed by gp140 and gp120 (3.53 nM and 28.1 nM K_{D}), respectively) (FIG. 7A) (52).

The V2q family include bnAbs such PG09 and PG16 which recognize a quaternary β-sheets structure in the V2 apex (51). It was found that PG09 only bound to 1086.c SOSIP trimer with significant affinity (12.4 nM K_{D}) (FIGS. 7A-B). No binding of PG16 to the three Env immunogens was observed (FIG. 7A).

Finally, the binding of the quaternary, trimer-specific PGT145 belonging to the V2qt family was analyzed (51). The PGT145 showed significant affinity only for the 1086.c SOSIP trimer (1.22 nM K_{D}) (FIGS. 7A-B). Both 1086.c gp140 an gp120 envelope proteins showed no detectable interaction with PGT145 (FIG. 7A).

### Neutralization Assay Based on In Vitro TMZ-bl Infection

A comprehensive neutralization profile of the important primary isolate Transmitted/Founder (T/F) clade C HIV-1strain C, 1086.c was determined. The ability of 27 broadly neutralizing and non-neutralizing antibodies to neutralize the 1086.c pseudotyped virus in vitro using the TMZ-bl neutralization assay was tested. For comparison, the bg505 virus with T332N mutation was used. Overall the 1086.c pseudotyped virus showed higher resistance to neutralization. Of the 25 bnAbs analyzed only seven antibodies neutralized the 1086.c virus (3BNC117, N6, VRC-31, HJ16, CH106, VRC01 and PGT121). Of the seven neutralizing antibodies, six were specific for the CD4 bs. PGT121 that binds to the Glycan Patch/V3 neutralized the 1086.c virus effectively (IC50<1 ng/ml). The BG505 T332N showed a more sensitive neutralization profile. The BG505 virus was neutralized by bnAbs belonging to all antibody groups (PG09, PGT145, PG16, 3BNC117, N6, VRC-CH31, VRC01, VRC03, PGT121, 10-1074, 2G12, PGT126 and PGT128) (FIG. 8). The neutralization potency of the chimeric antibody CD4-IgG2 which showed ability to neutralize only the 1086.c virus with an IC50 of 1.72 μg/ml was also assessed. As expected. the non-neutralizing mAb F105 did not neutralize either virus.

### DISCUSSION

There are several lines of evidence to support that an HIV-1 envelope glycoprotein-based vaccine that can elicit broadly neutralizing antibodies (bnAbs) or anti-V2 apex antibodies that mediate ADCC can be effective against HIV. A novel HIV-1 transmitted-founder (T/F) clade C 1086.c SOSIP gp140 trimer as a vaccine immunogen was generated. In a comprehensive antigenicity study, the 1086.c SOSIP trimer bound with considerable affinity to 16 of 19 bnAbs that recognize conserved epitopes in the V2 apex, CD4 binding site, and V3/glycan patch. The 1086.c SOSIP trimer overall exhibited a complementary antigenicity profile with BG505 SOSIP.664 trimer, but a similar antigenicity profile for certain bnAbs. Distinct from previously constructed trimers, the 1086.c SOSIP trimer displayed potentially protective epitopes in the V2 apex that were recognized with high affinity by functional RV144-related V2p antibodies. Interestingly, the PG09 bnAb as well as the RV144-related CH58 and CH59 antibodies bound strongly to the 1086.c SOSIP trimer, suggesting that the trimer contains a dynamic V2 apex structure. The observed preferential binding of anti-V2 apex bnAbs including the trimer specific PGT145 as well as V2p antibodies to the 1086.c SOSIP trimer over the 1086.c gp120 and gp140 non-trimer counterparts underscores the fundamental role of the native trimer structure in the proper presentation of not only bnAb epitopes but also potentially protective V2p epitopes. Fine structural analysis of the 1086.c SOSIP trimer's dynamic V2 apex and preclinical assessment of the novel trimer's ability to elicit bnAb and potentially protective non-neutralizing anti-V2 apex antibody responses are warranted.

Soluble native-like trimers mimic the true trimeric conformation of the HIV-1 envelope glycoproteins on the virion surface, and they contain bnAb epitopes in the V2 apex, CD4bs, V3/glycan patch, and gp120/gp41 interface (67). Because of these structural and antigenic features, trimers are considered viable immunogens for potentially eliciting broadly neutralizing antibody responses. In this study, a novel HIV-1 gp140 SOSIP trimer from the T/F tier 2 clade C 1086.c virus was generated because T/F clade C viruses are circulating viruses that are responsible for the majority of HIV transmission in Sub-Saharan Africa and over 50% of HIV infections globally (44). The 1086.c gp140 SOSIP envelope glycoproteins were produced in good quantity and purity. Importantly, biochemical and structural analyses revealed that the 1086.c gp140 SOSIP envelope glycoproteins were stable native-like trimers.

An antigenicity study of the clade C 1086.c SOSIP trimer was performed and compared to the clade A BG505 SOSIP.664 trimer. The binding of both native-like trimers to 19 bnAbs recognizing the V2 apex, CD4 bs, V3/glycan patch and gp120/gp41 interface were assessed. The majority of bnAbs tested and those with the most breadth and potency, including VRC01, N6, PG09, 3BNC117 recognized both trimers (70). In some instances, certain bnAbs such as PGT145, N6, VRC01, 3869, 447-52D bound more strongly to 1086.c SOSIP trimer. By comparison, PG09, 3BNC117, HJ16, CH106, PGT126, PGT128, 39F bound better to the BG505 SOSIP.664 trimer. Notable differences in binding to bnAbs were also observed. PG16, VRC03, 10-1074 and 35022 recognized the BG505 SOSIP.664 trimer but not the 1086.c SOSIP trimer. Conversely, the 697-30D and Ig1 B12 recognized the 1086.c SOSIP trimer but not the BG505 SOSIP.664 trimer. In addition to bnAbs, the binding of potentially protective non-neutralizing antibodies using the RV144 trial vaccinee-isolated CH58 mAb was also assessed (48). Interestingly, the CH58 mAb preferentially bound with high affinity to the 1086.c SOSIP trimer but not to BG505 SOSIP.664. Altogether, these results demonstrate that both 1086.c SOSIP or the BG505 SOSIP.664 trimers share certain bnAb epitopes, but overall, they have a complementary anti-HIV antibody-binding profile in that some important bnAb and potentially protective non-neutralizing antibody (CH58) antibodies bind preferentially to one or the other trimer.

The V2 apex is an important region of the ENV trimer protein because it is recognized by three bnAbs groups (Vi, V2q and V2qt) and contain a linear epitope recognized by the ADCC-inducing antibodies class (V2p) that correlated with the protection observed in the Thai vaccine clinical trial RV144 (47, 48, 52). Not previously seen with soluble trimers, the 1086.c SOSIP trimer was not only recognized by anti-V2 apex bnAbs, but also by the RV144-related CH58 mAb. V2 apex antigenicity analysis comparing the 1086.c trimer to the 1086.c gp140 and gp140 immunogens expectedly revealed that 1086.c SOSIP trimer was recognized more broadly and with markedly higher affinity by the V2 apex antibodies compared to the non-trimeric counterparts. The 1086.c SOSIP trimer was recognized by all the V2 apex-specific antibodies that were tested except for the V2q bnAb PG16. The 1086.c SOSIP trimer exhibited strong affinity to PGT145 bnAb and significant affinity to PG09 (a V2q bnAb), while the gp120 and gp140 non-trimer counterparts showed no reactivity to these bnAbs. The preferential binding of the trimer specific PGT145 bnAb to the 1086.c SOSIP trimer suggests that the 1086.c SOSIP trimer presents key epitopes that have become hallmarks of the native trimer (35, 71, 72).

Post-hoc analysis of the RV144 trial revealed the importance of inducing anti-V1-V2 antibodies. specifically the V2p class of antibodies. for protection. Functional analysis of V2p antibodies isolated from RV144 vaccinees suggested that ADCC may play an important role in protection against HIV (48). While anti-V2 apex bnAbs were capable of binding significantly to the 1086.c SOSIP trimer, the V2p antibodies CH58, CH59, HG107, and HG120 bound with high affinity to the 1086.c SOSIP trimer as well, reinforcing the uniqueness of this trimer compared to previously reported trimers (48). Binding of the V2p antibodies to the trimer was markedly stronger than to the 1086.c gp120 and gp140 counterparts suggesting that the trimer conformation of the V2 apex is the preferred structure for optimal binding of V2p antibodies.

V2p antibodies such as CH58 were induced in RV144 vaccinee, but more recently an individual infected with the Clade C virus developed V2p antibodies with ADCC activity of significant breadth (73, 74). In addition, the V2p antibody CAP228 which was isolated from the clade C-infected individual, was shown to bind to 1086.c Env (74). It was therefore not surprising that the 1086.c SOSIP clade C trimer was also found to contain V2p epitopes. The amino acid positions present in the antibody binding site recognized by CAP228 mAbs are among the most conserved in V2 across globally circulating strains in the Los Alamos National Laboratory HIV-1 Env database and are likely important determinants of the cross-reactivity for V2-binding antibodies (74). This broad recognition by CAP228 mAbs translated into significant breadth of ADCC activity against globally prevalent V2 immunotypes (74). The CAP228 mAbs was found to be similar to V2-directed antibodies from the RV144 vaccine trial that exhibited restricted light chain usage, but interestingly, a CAP228 antibody lineage was shown to use a third V2-reactive light chain gene (74). This additional light chain gene used by a V2p antibody suggest a larger antibody repertoire, and therefore V2p antibodies may be easier to elicit by vaccination than previously recognized. The extent the V2p epitopes present on the 1086.c SOSIP trimer is conserved and whether the 1086.c SOSIP trimer could elicit V2p antibodies that have broad ADCC activity against circulating global HIV-1 isolates can be examined further.

The 1086.c SOSIP trimer is recognized by both potentially protective V2p antibodies and anti-V2 apex bnAbs, which suggests that the 1086.c trimer V2 apex has a unique and interesting structure. The binding of PG09, CH58 and CH59 to the V2 apex of the 1086.c SOSIP trimer has never been reported for other native-like trimers (FIGS. 7A-B). This is likely because CH58 recognizes region 167-176 as an α-helix, and 177-181 as coiled loop, while CH59 binds region 168-173 as a coil, and 174-176 as a short 310 helix (48). By comparison PG09 recognizes region 167-181 as a β-strand (75). Consistent to the previous observation by Liao et al., the three binding conformations in the V2 apex likely co-exist or are interchangeable in the 1086.c SOSIP trimer (48).

Due to the extraordinarily high genetic diversity among HIV-1 clades, the selection of a particular HIV-1 Env sequence for creating a trimer vaccine that will be effective is somewhat complex. The geographical distribution of the different HIV clades led to the most realistic approach to vaccine design which is to use a clade-specific vaccine. Indeed, it was found that notable differences in antigenicity amongst the trimers from different clades (clade A, B and C), likely reflect significant interclade sequence divergence (27-35). However, given that some potentially protective epitopes recognized by bnAbs were found to be conserved on the envelope trimer across many different strains and clades, a universal vaccine is conceivable. With respect to potentially protective V2 apex epitopes seen in the RV144 trial and recently in natural infection, conservation of these important epitopes is just beginning to be realized (74). The impact of interclade genetic differences on trimer antigenicity and ultimately on vaccine effectiveness need to be addressed for the proper design of highly effective trimer vaccine immunogens.

In summary (Example 1), a highly antigenic novel T/F clade C 1086.c SOSIP Env trimer that contains potentially protective anti-HIV-1 epitopes has been constructed. The 1086.c SOSIP trimer is a unique immunogen that contains not only important bnAb epitopes, but also potentially protective epitopes recognized by V2p antibodies. Since soluble trimers were previously shown to only elicit autologous tier 2 neutralizing antibodies, it needs to be determined whether the 1086.c SOSIP trimer will elicit homologous and heterologous Tier 2 bnAb responses. Similar to the BG505 SOSIP.664 trimer, further modifications of the 1086.c SOSIP trimer may be beneficial to engage bnAb precursors (26). The complementary antigenicity profile of both trimers supports including both immunogens in a multivalent vaccine strategy designed to engage bnAb precursors to broaden neutralizing antibody responses. Finally, whether 1086.c SOSIP trimer immunogen is capable of eliciting V2p antibodies with broad ADCC activity can be assessed. Future 1086.c SOSIP trimer vaccine immunogenicity and efficacy testing in laboratory animals is forthcoming.

### EXAMPLE 2

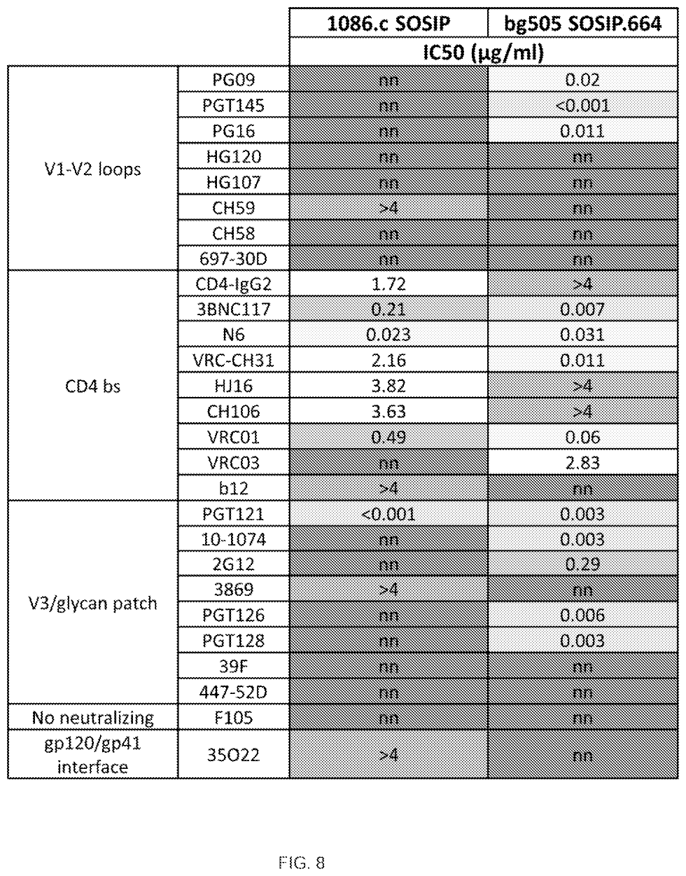

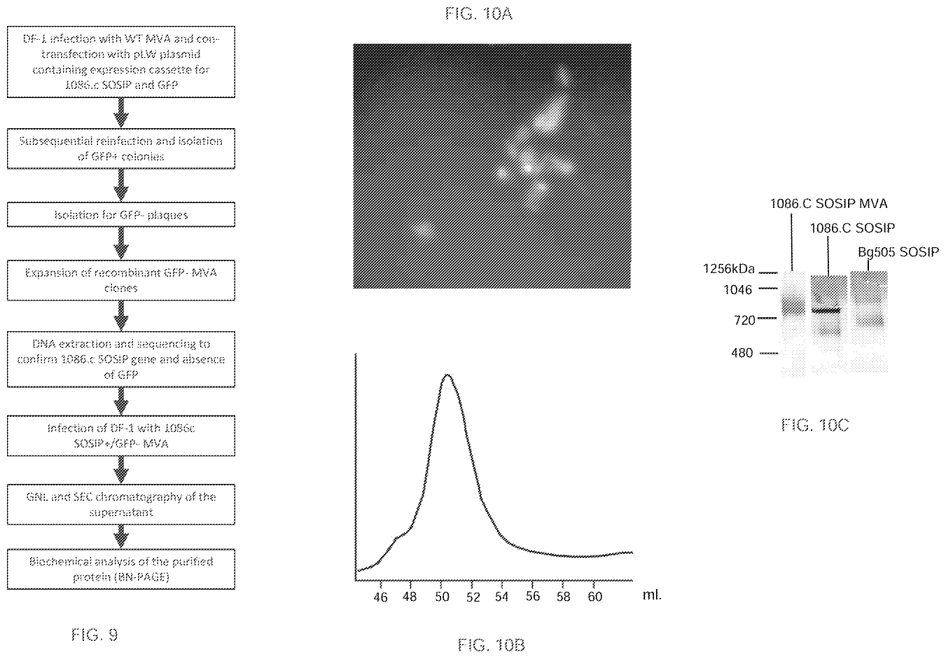

### Materials and Methods (Flow Chart—FIG. 9)

DF-1 cells were transfected using Lipofectamine 2000 (Thermofisher, Rockford, IL USA). 50,000 cells were seeded in a 6-well plate and cultured overnight in 2 ml DMEM 10% FBS. The following day, the cells were transfected using 4 μg of pLW76-1086c SOSIP plasmid. The pLW76 expression cassette is flanked by two homologous sequences that promote homology recombination in the MVA wild type virus resulting in the substitution of the region comprise between position 142,591 and position 145,229 of the virus genome. Cells were incubated overnight and then the medium replaced with fresh DMEM, 10% FBS, 2% PEN/STREP. 1 μl of MVA wild type was then added to the culture. The culture medium was replaced with 2.5% methyl cellulose prepared in DMEM, 10% FBS and 2% PEN/STREP.

After 48-72 hours, the recombinant virus was isolated from the GFP positive colonies under fluorescent microscope using a pipet tip (FIG. 10A). The recombinant MVA was resuspended in 50 μl DMEM medium. The rMVA was used to infect fresh DF-1 cultures and 3-5 passages of isolation were performed. Contamination with wild type MVA was assessed by sequencing.

Once a non-contaminated rMVA-1086c SOSIP was isolated, GFP negative clones picked. Absence of GFP gene was assessed both by fluorescence and sequencing. The rMVA 1086c SOSIP GFP-virus was then used to infect DF-1 cells in 10 cm culture dish. After 5 days, the supernatant was collected, and the soluble protein was purified by GNL chromatography and SEC. BN page Coomassie was used to determine protein conformation (FIGS. 10B-C).

GNL and SEC purification: The supernatant was processed through a Galanthus Nivalis Lectin Colum (GNL) (Vector laboratories, Burlingame, CA) by gravity. The column was washed with 2 volumes of washing buffer (0.5M NaCl in PBS) and then rinsed with PBS. After closing the column stopcock, 2 volumes of Methyl α-D-mannopyranoside 1M (Cat. #M6882 Sigma Aldrich, St. Louis, MO) in PBS/pH7.4 were added to the column and incubated for 1 minute. The eluted sample was collected in a storage bottle filled with 2 volumes of TN-75 (TRIS 20 nM; NaCl 75 mM; pH8.0) and subsequently concentrated to a final volume of 1 ml using Amicon® Ultra-15 100 KDa Centrifugal Filter Units (Millipore, Burlinghton, MA).
SEC chromatography: The AKTA 100 Explore FPLC was equipped with a HighLoad 16/600 Superdex 200 pg Column (GE healthcare, Chicago, IL). The FPLC system was equilibrated with TN-75 (TRIS 20 nM; NaCl 75 mM; pH8.0) until stabilization of 215 nm and 280 nm absorbance signal was obtained. The protein concentrated from GNL chromatography was injected and processed with a flow rate of 0.7 ml/min and 2-5 ml fractions were collected. The trimer-specific fraction was then pooled and concentrated to a final 500 ul volume using Amicon® Ultra-15 100 KDa Centrifugal Filter Units (Millipore, Burlinghton, MA). The final protein sample was quantified by 280 nm absorbance using a nanodrop 2000 (ThermoFisher, Waltham, MA).
BN-Page: The rMVA purified protein (2 ug) were resuspended in Native PAGE sample buffer and loaded into Native PAGE 4-16% Bis-Tris gel (Invitrogen, Carlsbad, CA). Electrophoresis was conducted at 120 V for 2 hours in a light blue solution containing Native PAGE running buffer and Native PAGE cathode additive (Invitrogen, Carlsbad, CA). The HIV proteins were then stained with GelCode Blue Safe Protein Stain (Thermo Scientific, Rockford, IL. USA) for 1-1.5 hours and analyzed using the Innotech FluorchemQ image III system. NativeMark Protein Standard (Invitrogen, Carlsbad, CA) was used as a molecular weight marker.

### RESULTS

MVA has shown a potent vaccine vector to induce immunity against Smallpox but also other infectious diseases such as avian influenza H5N1 and Ebola virus (Volz). Along with simplicity of production, strict virus specific gene expression, large DNA capacity, wide cell tropism and short life in the host made this system a strong candidate as vaccine vector.

After several adaptation passages in Chicken, MVA lost its virulency in mammalian making it safe and armless. The inactivation of the virus in mammalian is observed at the stage of the packaging so preserving the protein expression. These characteristics makes the MVA an interesting candidate vaccine vector for the ectopic expression of immunogens of interest.

MVA has shown to be effective in eliciting the production and release of native-like soluble bg505 SOSIP in solution and the induction of autologous nAbs in rabbit (Capucci).

1086c protein was detected in the supernatant of infected 293, cells as shown by both SEC and BN PAGE followed by Coomassie stain (FIGS. 10B-C), in this vaccine protocol.

### CONCLUSION

All patents and publications mentioned in this specification are indicative of the levels of those skilled in the art to which the invention pertains. All patents and publications are herein incorporated by reference to the same extent as if each individual publication were specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is adapted to carry out the objectives and obtain the ends and advantages mentioned, as well as those inherent therein. The immunogens, vaccines, and methods described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention, which are obvious to those skilled in the art, are intended to be within the scope of the invention.

### REFERENCES

1. Rerks-Ngarm S, Pitisuttithum P, Nitayaphan S, Kaewkungwal J, Chiu J, Paris R, Premsri N, Namwat C, de Souza M, Adams E, Benenson M, Gurunathan S, Tartaglia J, McNeil J G, Francis D P, Stablein D, Birx D L, Chunsuttiwat S, Khamboonruang C, Thongcharoen P, Robb M L, Michael N L, Kunasol P, Kim J H. 2009. Vaccination with ALVAC and AIDSVAX to prevent HIV-1 infection in Thailand. N Engl J Med 361:2209-20.
2. Haynes B F, Gilbert P B, McElrath M J, Zolla-Pazner S, Tomaras G D, Alam S M, Evans D T, Montefiori D C, Karnasuta C, Sutthent R, Liao H X, DeVico A L, Lewis G K, Williams C, Pinter A, Fong Y, Janes H, DeCamp A, Huang Y, Rao M, Billings E, Karasavvas N, Robb M L, Ngauy V, de Souza M S, Paris R, Ferrari G, Bailer R T, Soderberg K A, Andrews C, Berman P W, Frahm N, De Rosa S C, Alpert M D, Yates N L, Shen X, Koup R A, Pitisuttithum P, Kaewkungwal J, Nitayaphan S, Rerks-Ngarm S, Michael N L, Kim J H. 2012. Immune-correlates analysis of an HIV-1 vaccine efficacy trial. N Engl J Med 366: 1275-86.
3. Chung A W, Ghebremichael M, Robinson H, Brown E, Choi I, Lane S, Dugast A S, Schoen M K, Rolland M, Suscovich T J, Mahan A E, Liao L, Streeck H, Andrews C, Rerks-Ngarm S, Nitayaphan S, de Souza M S, Kaewkungwal J, Pitisuttithum P, Francis D, Michael N L, Kim J H, Bailey-Kellogg C, Ackerman M E, Alter G. 2014. Polyfunctional Fc-effector profiles mediated by IgG subclass selection distinguish RV144 and VAX003 vaccines. Sci Transl Med 6:228ra38.
4. Tay M Z, Liu P, Williams L D, McRaven M D, Sawant S, Gurley T C, Xu T T, Dennison S M, Liao H X, Chenine A L, Alam S M, Moody M A, Hope T J, Haynes B F, Tomaras G D. 2016. Antibody-Mediated Internalization of Infectious HIV-1 Virions Differs among Antibody Isotypes and Subclasses. PLOS Pathog 12:e1005817.
5. O'Connell R J, Excler J L. 2013. HIV vaccine efficacy and immune correlates of risk. Curr HIV Res 11:450-63.
6. Corey L, Gilbert P B, Tomaras G D, Haynes B F, Pantaleo G, Fauci A S. 2015. Immune correlates of vaccine protection against HIV-1 acquisition. Sci Transl Med 7:310rv7.
7. Kim J H, Excler J L, Michael N L. 2015. Lessons from the RV144 Thai phase III HIV-1 vaccine trial and the search for correlates of protection. Annu Rev Med 66:423-37.
8. Liao H X, Bonsignori M, Alam S M, Mclellan J S, Tomaras G D, Moody M A, Kozink D M, Hwang K K, Chen X, Tsao C Y, Liu P, Lu X, Parks R J, Montefiori D C, Ferrari G, Pollara J, Rao M, Peachman K K, Santra S, Letvin N L, Karasavvas N, Yang Z Y, Dai K, Pancera M, Gorman J, Wiehe K, Nicely N I, Rerks-Ngarm S, Nitayaphan S, Kaewkungwal J, Pitisuttithum P, Tartaglia J, Sinangil F, Kim J H, Michael N L, Kepler T B, Kwong P D, Mascola J R, Nabel G J, Pinter A, Zolla-Pazner S, Haynes B F. 2013. Vaccine induction of antibodies against a structurally heterogeneous site of immune pressure within HIV-1 envelope protein variable regions 1 and 2. Immunity 38:176-86.
9. Perez L G, Martinez D R, deCamp A C, Pinter A, Berman P W, Francis D, Sinangil F, Lee C, Greene K, Gao H, Nitayaphan S, Rerks-Ngarm S, Kaewkungwal J, Pitisuttithum P, Tartaglia J, O'Connell R J, Robb M L, Michael N L, Kim J H, Gilbert P. Montefiori D C. 2017. VIV2-specific complement activating serum IgG as a correlate of reduced HIV-1 infection risk in RV144. PloS one 12:e0180720-e0180720.
10. Su B, Moog C. 2014. Which Antibody Functions are Important for an HIV Vaccine? Front Immunol 5:289.
11. Forthal D, Hope T J, Alter G. 2013. New paradigms for functional HIV-specific nonneutralizing antibodies. Curr Opin HIV AIDS 8:393-401.
12. Ndung'u T, Weiss R A. 2012. On HIV diversity. AIDS 26.
13. Doria-Rose N A, Klein R M, Manion M M, O'Dell S, Phogat A, Chakrabarti B, Hallahan C W, Migueles S A, Wrammert J, Ahmed R, Nason M, Wyatt R T, Mascola J R, Connors M. 2009. Frequency and phenotype of human immunodeficiency virus envelope-specific B cells from patients with broadly cross-neutralizing antibodies. J Virol 83:188-99.
14. Gray E S, Madiga M C, Hermanus T, Moore P L, Wibmer C K, Tumba N L, Werner L, Mlisana K. Sibeko S, Williamson C, Abdool Karim S S, Morris L. 2011. The neutralization breadth of HIV-1 develops incrementally over four years and is associated with CD4+ T cell decline and high viral load during acute infection. J Virol 85:4828-40.
15. van Gils M J, Euler Z, Schweighardt B, Wrin T, Schuitemaker H. 2009. Prevalence of cross-reactive HIV-1-neutralizing activity in HIV-1-infected patients with rapid or slow disease progression. Aids 23:2405-14.
16. Mascola J R, Stiegler G, VanCott T C, Katinger H, Carpenter C B, Hanson C E, Beary H, Hayes D, Frankel S S, Birx D L, Lewis M G. 2000. Protection of macaques against vaginal transmission of a pathogenic HIV-1/SIV chimeric virus by passive infusion of neutralizing antibodies. Nat Med 6:207-10.
17. Parren P W, Marx P A, Hessell A J, Luckay A, Harouse J, Cheng-Mayer C, Moore J P, Burton D R. 2001. Antibody protects macaques against vaginal challenge with a pathogenic R5 simian/human immunodeficiency virus at serum levels giving complete neutralization in vitro. J Virol 75:8340-7.
18. Shibata R, Igarashi T, Haigwood N, Buckler-White A, Ogert R, Ross W, Willey R, Cho M W, Martin M A. 1999. Neutralizing antibody directed against the HIV-1 envelope glycoprotein can completely block HIV-1/SIV chimeric virus infections of macaque monkeys. Nat Med 5:204-10.
19. Hessell A J, Rakasz E G, Poignard P, Hangartner L, Landucci G, Forthal D N, Koff W C, Watkins D I, Burton D R. 2009. Broadly neutralizing human anti-HIV antibody 2G12 is effective in protection against mucosal SHIV challenge even at low serum neutralizing titers. PLOS Pathog 5:e1000433.
20. Hessell A J, Poignard P, Hunter M, Hangartner L, Tehrani D M, Bleeker W K, Parren P W, Marx P A, Burton D R. 2009. Effective, low-titer antibody protection against low-dose repeated mucosal SHIV challenge in macaques. Nat Med 15:951-4.
21. Burton D R, Hangartner L. 2016. Broadly Neutralizing Antibodies to HIV and Their Role in Vaccine Design. Annu Rev Immunol 34:635-59.
22. Julien J -P, Cupo A, Sok D, Stanfield R L, Lyumkis D, Deller M C, Klasse P -J, Burton D R,. Sanders R W, Moore J P, Ward A B, Wilson I A. 2013. Crystal structure of a soluble cleaved HIV-1 envelope trimer. Science (New York, NY) 342:1477-1483.
23. Lyumkis D, Julien J -P, de Val N, Cupo A, Potter C S, Klasse P -J, Burton D R, Sanders R W, Moore J P, Carragher B, Wilson I A, Ward A B. 2013. Cryo-EM structure of a fully glycosylated soluble cleaved HIV-1 envelope trimer. Science (New York, NY) 342:1484-1490.
24. Pauthner M, Havenar-Daughton C, Sok D, Nkolola J P, Bastidas R, Boopathy A V, Carnathan D G, Chandrashekar A, Cirelli K M, Cottrell C A, Eroshkin A M, Guenaga J, Kaushik K, Kulp D W, Liu J, McCoy L E, Oom A L, Ozorowski G, Post K W, Sharma S K, Steichen J M, de Taeye S W, Tokatlian T, Torrents de la Peña A, Butera S T, LaBranche C C, Montefiori D C, Silvestri G, Wilson I A, Irvine D J, Sanders R W, Schief W R, Ward A B, Wyatt R T, Barouch D H, Crotty S, Burton D R. 2017. Elicitation of Robust Tier 2 Neutralizing Antibody Responses in Nonhuman Primates by HIV Envelope Trimer Immunization Using Optimized Approaches. Immunity 46:1073-1088.e6.
25. Sanders R W, Derking R, Cupo A, Julien J P, Yasmeen A, de Val N, Kim H J, Blattner C, de la Peña A T, Korzun J, Golabek M, de Los Reyes K, Ketas T J, van Gils M J, King C R, Wilson I A, Ward A B, Klasse P J, Moore J P. 2013. A next-generation cleaved, soluble HIV-1 Env trimer, BG505 SOSIP.664 gp140, expresses multiple epitopes for broadly neutralizing but not non-neutralizing antibodies. PLOS Pathog 9:e1003618.
26. Medina-Ramirez M, Garces F, Escolano A, Skog P, de Taeye S W, Del Moral-Sanchez I, McGuire A T, Yasmeen A, Behrens A -J, Ozorowski G, van den Kerkhof T L G M, Freund N T, Dosenovic P, Hua Y, Gitlin A D, Cupo A, van der Woude P, Golabek M, Sliepen K, Blane T, Kootstra N, van Breemen M J, Pritchard L K, Stanfield R L, Crispin M, Ward A B, Stamatatos L, Klasse P J, Moore J P, Nemazee D, Nussenzweig M C, Wilson I A, Sanders R W. 2017. Design and crystal structure of a native-like HIV-1 envelope trimer that engages multiple broadly neutralizing antibody precursors in vivo. Journal of Experimental Medicine 214:2573-2590.
27. Torrents de la Peña A, de Taeye S W, Sliepen K, LaBranche C C, Burger J A, Schermer E E, Montefiori D C, Moore J P, Klasse P J, Sanders R W. 2018. Immunogenicity in Rabbits of HIV-1 SOSIP Trimers from Clades A, B, and C, Given Individually, Sequentially, or in Combination. J Virol 92.
28. Ananthaswamy N, Fang Q, AlSalmi W, Jain S, Chen Z, Klose T, Sun Y, Liu Y, Mahalingam M, Chand S, Tovanabutra S, Robb M L, Rossmann M G, Rao V B. 2019. A sequestered fusion peptide in the structure of an HIV-1 transmitted founder envelope trimer. Nature communications 10:873-873.
29. de Taeye S W, Ozorowski G, Torrents de la Peña A, Guttman M, Julien J -P, van den Kerkhof T L G M, Burger J A, Pritchard L K, Pugach P, Yasmeen A, Crampton J, Hu J, Bontjer I, Torres J L, Arendt H, DeStefano J, Koff W C, Schuitemaker H, Eggink D, Berkhout B, Dean H, LaBranche C, Crotty S, Crispin M, Montefiori D C, Klasse P J, Lee K K, Moore J P, Wilson I A, Ward A B, Sanders R W. 2015. Immunogenicity of Stabilized HIV-1 Envelope Trimers with Reduced Exposure of Non-neutralizing Epitopes. Cell 163:1702-1715.
30. Julien J P, Lee J H, Ozorowski G, Hua Y, Torrents de la Peña A, de Taeye S W, Nieusma T, Cupo A, Yasmeen A, Golabek M, Pugach P, Klasse P J, Moore J P, Sanders R W, Ward A B, Wilson I A. 2015. Design and structure of two HIV-1 clade C SOSIP.664 trimers that increase the arsenal of native-like Env immunogens. Proc Natl Acad Sci USA 112:11947-52.
31. Pugach P. Ozorowski G, Cupo A, Ringe R, Yasmeen A, de Val N, Derking R, Kim H J, Korzun J, Golabek M, de Los Reyes K, Ketas T J, Julien J P, Burton D R, Wilson I A, Sanders R W, Klasse P J, Ward A B, Moore J P. 2015. A native-like SOSIP.664 trimer based on an HIV-1 subtype B env gene. J Virol 89:3380-95.
32. Feng Y, Tran K, Bale S, Kumar S, Guenaga J, Wilson R, de Val N, Arendt H, DeStefano J, Ward A B, Wyatt R T. 2016. Thermostability of Well-Ordered HIV Spikes Correlates with the Elicitation of Autologous Tier 2 Neutralizing Antibodies. PLOS pathogens 12:e1005767-e1005767.
33. Klasse P J, LaBranche C C, Ketas T J, Ozorowski G, Cupo A, Pugach P, Ringe R P, Golabek M, van Gils M J, Guttman M, Lee K K, Wilson I A, Butera S T, Ward A B, Montefiori D C. Sanders R W, Moore J P. 2016. Sequential and Simultaneous Immunization of Rabbits with HIV-1 Envelope Glycoprotein SOSIP.664 Trimers from Clades A, B and C. PLOS Pathog 12:e1005864.
34. Guenaga J, de Val N, Tran K, Feng Y, Satchwell K, Ward A B, Wyatt R T. 2015. Well-ordered trimeric HIV-1 subtype B and C soluble spike mimetics generated by negative selection display native-like properties. PLOS Pathog 11:e1004570.
35. Ringe R P, Yasmeen A, Ozorowski G, Go E P, Pritchard L K, Guttman M, Ketas T A, Cottrell C A, Wilson I A, Sanders R W, Cupo A, Crispin M, Lee K K, Desaire H, Ward A B, Klasse P J, Moore J P. 2015. Influences on the Design and Purification of Soluble, Recombinant Native-Like HIV-1 Envelope Glycoprotein Trimers. J Virol 89:12189-210.
36. Sliepen K. Han B W, Bontjer I, Mooij P, Garces F, Behrens A -J, Rantalainen K, Kumar S, Sarkar A, Brouwer P J M, Hua Y, Tolazzi M, Schermer E, Torres J L, Ozorowski G, van der Woude P, de la Peña A T, van Breemen M J, Camacho-Sánchez J M, Burger J A, Medina-Ramírez M, González N, Alcami J, LaBranche C, Scarlatti G, van Gils M J, Crispin M, Montefiori D C, Ward A B, Koopman G, Moore J P, Shattock R J, Bogers W M, Wilson I A, Sanders R W. 2019. Structure and immunogenicity of a stabilized HIV-1 envelope trimer based on a group-M consensus sequence. Nature Communications 10:2355.
37. Bale S, Martiné A, Wilson R, Behrens A J, Le Fourn V, de Val N, Sharma S K, Tran K, Torres J L, Girod P A, Ward A B, Crispin M, Wyatt R T. 2018. Cleavage-Independent HIV-1 Trimers From CHO Cell Lines Elicit Robust Autologous Tier 2 Neutralizing Antibodies. Front Immunol 9:1116.
38. Georgiev I S, Joyce M G, Yang Y, Sastry M, Zhang B, Baxa U, Chen R E, Druz A, Lees C R, Narpala S, Schön A, Van Galen J, Chuang G Y, Gorman J, Harned A, Pancera M, Stewart-Jones G B, Cheng C, Freire E, McDermott A B, Mascola J R, Kwong P D. 2015. Single-Chain Soluble BG505.SOSIP gp140 Trimers as Structural and Antigenic Mimics of Mature Closed HIV-1 Env. J Virol 89:5318-29.
39. Kong L, He L, de Val N, Vora N, Morris C D, Azadnia P, Sok D, Zhou B, Burton D R, Ward A B, Wilson I A, Zhu J. 2016. Uncleaved prefusion-optimized gp140 trimers derived from analysis of HIV-1 envelope metastability. Nat Commun 7:12040.
40. Sharma S K, de Val N, Bale S, Guenaga J, Tran K, Feng Y, Dubrovskaya V, Ward A B, Wyatt R T. 2015. Cleavage-independent HIV-1 Env trimers engineered as soluble native spike mimetics for vaccine design. Cell Rep 11:539-50.
41. Sattentau Q J, Moore J P. 1995. Human immunodeficiency virus type 1 neutralization is determined by epitope exposure on the gp120 oligomer. The Journal of experimental medicine 182:185-196.
42. Binley J M, Sanders R W, Clas B, Schuelke N, Master A, Guo Y, Kajumo F, Anselma D J, Maddon P J, Olson W C, Moore J P. 2000. A recombinant human immunodeficiency virus type 1 envelope glycoprotein complex stabilized by an intermolecular disulfide bond between the gp120 and gp41 subunits is an antigenic mimic of the trimeric virion-associated structure. Journal of virology 74:627-643.
43. de Taeye S W, Moore J P, Sanders R W. 2016. HIV-1 Envelope Trimer Design and Immunization Strategies To Induce Broadly Neutralizing Antibodies. Trends in immunology 37:221-232.
44. Hemelaar J, Gouws E, Ghys P D, Osmanov S. 2011. Global trends in molecular 44. epidemiology of HIV-1 during 2000-2007. Aids 25:679-89.
45. Liao H -X, Tsao C -Y, Alam S M, Muldoon M, Vandergrift N, Ma B -J, Lu X, Sutherland L L, Scearce R M, Bowman C, Parks R, Chen H, Blinn J H, Lapedes A, Watson S, Xia S -M, Foulger A, Hahn B H, Shaw G M, Swanstrom R, Montefiori D C, Gao F, Haynes B F, Korber B. 2013. Antigenicity and immunogenicity of transmitted/founder, consensus, and chronic envelope glycoproteins of human immunodeficiency virus type 1. Journal of virology 87:4185-4201.
46. Alexander J, Mendy J, Vang L, Avanzini J B, Garduno F, Manayani D J, Ishioka G, Farness P, Ping L -H, Swanstrom R, Parks R, Liao H -X, Haynes B F, Montefiori D C, LaBranche C, Smith J, Gurwith M, Mayall T. 2013. Pre-clinical development of a recombinant, replication-competent adenovirus serotype 4 vector vaccine expressing HIV-1 envelope 1086 clade C. PloS one. 8(12):e82380. doi: 10.1371/journal.pone.0082380.
47. Haynes B F, Gilbert P B, McElrath M J, Zolla-Pazner S, Tomaras G D, Alam S M, Evans D T, Montefiori D C, Karnasuta C, Sutthent R, Liao H -X, DeVico A L, Lewis G K, Williams C, Pinter A, Fong Y, Janes H, DeCamp A, Huang Y, Rao M, Billings E, Karasavvas N, Robb M L, Ngauy V, de Souza M S, Paris R, Ferrari G, Bailer R T, Soderberg K A, Andrews C, Berman P W, Frahm N, De Rosa S C, Alpert M D, Yates N L, Shen X, Koup R A, Pitisuttithum P, Kaewkungwal J, Nitayaphan S, Rerks-Ngarm S, Michael N L, Kim J H. 2012. Immune-Correlates Analysis of an HIV-1 Vaccine Efficacy Trial. New England Journal of Medicine 366:1275-1286.
48. Liao H -X, Bonsignori M, Alam S M, Mclellan J S, Tomaras G D, Moody M A, Kozink D M, Hwang K -K, Chen X, Tsao C -Y, Liu P, Lu X, Parks R J, Montefiori D C, Ferrari G, Pollara J, Rao M, Peachman K K, Santra S, Letvin N L, Karasavvas N, Yang Z -Y, Dai K, Pancera M, Gorman J, Wiehe K, Nicely N I, Rerks-Ngarm S, Nitayaphan S, Kaewkungwal J, Pitisuttithum P, Tartaglia J, Sinangil F, Kim J H, Michael N L, Kepler T B, Kwong P D, Mascola J R, Nabel G J, Pinter A, Zolla-Pazner S, Haynes B F. 2013. Vaccine induction of antibodies against a structurally heterogeneous site of immune pressure within HIV-1 envelope protein variable regions 1 and 2. Immunity 38:176-186.
49. Klasse P J, Depetris R S, Pejchal R, Julien J -P, Khayat R, Lee J H, Marozsan A J, Cupo A, Cocco N, Korzun J, Yasmeen A, Ward A B, Wilson I A, Sanders R W, Moore J P. 2013. Influences on trimerization and aggregation of soluble, cleaved HIV-1 SOSIP envelope glycoprotein. Journal of virology 87:9873-9885.
50. Walker L M, Phogat S K, Chan-Hui P Y, Wagner D, Phung P, Goss J L, Wrin T, Simek M D, Fling S, Mitcham J L, Lehrman J K, Priddy F H, Olsen O A, Frey S M, Hammond P W, Kaminsky S, Zamb T, Moyle M, Koff W C, Poignard P, Burton D R. 2009. Broad and potent neutralizing antibodies from an African donor reveal a new HIV-1 vaccine target. Science 326:285-9.
51. Walker L M, Huber M, Doores K J, Falkowska E, Pejchal R, Julien J-P, Wang S -K, Ramos A, Chan-Hui P -Y, Moyle M, Mitcham J L, Hammond P W, Olsen O A, Phung P, Fling S, Wong C -H, Phogat S, Wrin T, Simek M D, Principal Investigators P G, Koff W C, Wilson I A, Burton D R, Poignard P. 2011. Broad neutralization coverage of HIV by multiple highly potent antibodies. Nature 477:466-470).
52. Gorny M K, Moore J P, Conley A J, Karwowska S, Sodroski J, Williams C, Burda S, Boots L J, Zolla-Pazner S. 1994. Human anti-V2 monoclonal antibody that neutralizes primary but not laboratory isolates of human immunodeficiency virus type 1. Journal of Virology 68:8312.
53. Klein F, Gaebler C, Mouquet H, Sather D N, Lehmann C, Scheid J F, Kraft Z, Liu Y, Pietzsch J, Hurley A, Poignard P, Feizi T, Morris L, Walker B D, Fätkenheuer G, Seaman M S, Stamatatos L, Nussenzweig M C. 2012. Broad neutralization by a combination of antibodies recognizing the CD4 binding site and a new conformational epitope on the HIV-1 envelope protein. J Exp Med 209:1469-79.
54. Wu X, Yang Z Y, Li Y, Hogerkorp C M, Schief W R, Seaman M S, Zhou T, Schmidt S D, Wu L, Xu L, Longo N S, McKee K, O'Dell S, Louder M K, Wycuff D L, Feng Y, Nason M, Doria-Rose N, Connors M, Kwong P D, Roederer M, Wyatt R T, Nabel G J, Mascola J R. 2010. Rational design of envelope identifies broadly neutralizing human monoclonal antibodies to HIV-1. Science 329:856-61.
55. Barbas C F, 3rd, Björling E, Chiodi F, Dunlop N, Cababa D, Jones T M, Zebedee S L, Persson M A, Nara P L, Norrby E, et al. 1992. Recombinant human Fab fragments neutralize human type 1 immunodeficiency virus in vitro. Proc Natl Acad Sci USA 89:9339-43.
56. Burton D R, Pyati J, Koduri R, Sharp S J, Thornton G B, Parren P W, Sawyer L S, Hendry R M, Dunlop N, Nara P L, et al. 1994. Efficient neutralization of primary isolates of HIV-1 by a recombinant human monoclonal antibody. Science 266:1024-7.
57. Huang J, Kang B H, Ishida E, Zhou T, Griesman T, Sheng Z, Wu F, Doria-Rose N A, Zhang B, McKee K, O'Dell S, Chuang G Y, Druz. A, Georgiev I S, Schramm C A, Zheng A, Joyce M G, Asokan M, Ransier A, Darko S, Migueles S A, Bailer R T, Louder M K, Alam S M, Parks R, Kelsoe G, Von Holle T, Haynes B F, Douek D C, Hirsch V. Seaman M S, Shapiro L, Mascola J R, Kwong P D, Connors M. 2016. Identification of a CD4-Binding-Site Antibody to HIV that Evolved Near-Pan Neutralization Breadth. Immunity 45:1108-1121.
58. Corti D, Langedijk J P, Hinz. A, Seaman M S, Vanzetta F, Fernandez-Rodriguez. B M, Silacci C, Pinna D, Jarrossay D, Balla-Jhagjhoorsingh S, Willems B, Zekveld M J, Dreja H, O'Sullivan E, Pade C, Orkin C, Jeffs S A, Montefiori D C, Davis D, Weissenhorn W, Mcknight A, Heeney J L, Sallusto F, Sattentau Q J, Weiss R A, Lanzavecchia A. 2010. Analysis of memory B cell responses and isolation of novel monoclonal antibodies with neutralizing breadth from HIV-1-infected individuals. PLOS One 5:e8805.
59. Liao H -X, Lynch R, Zhou T, Gao F, Alam S M, Boyd S D, Fire A Z, Roskin K M, Schramm C A, Zhang Z, Zhu J, Shapiro L, Becker J, Benjamin B, Blakesley R, Bouffard G, Brooks S, Coleman H, Dekhtyar M, Gregory M, Guan X, Gupta J, Han J, Hargrove A, Ho S-1, Johnson T, Legaspi R, Lovett S, Maduro Q, Masiello C, Maskeri B, McDowell J, Montemayor C, Mullikin J, Park M, Riebow N, Schandler K, Schmidt B, Sison C, Stantripop M, Thomas J, Thomas P, Vemulapalli M, Young A, Mullikin J C, Gnanakaran S, Hraber P, Wiehe K, Kelsoe G, Yang G, et al. 2013. Co-evolution of a broadly neutralizing HIV-1 antibody and founder virus. Nature 496:469-476.
60. Trkola A, Purtscher M, Muster T, Ballaun C, Buchacher A, Sullivan N, Srinivasan K, Sodroski J, Moore J P, Katinger H. 1996. Human monoclonal antibody 2G12 defines a distinctive neutralization epitope on the gp120 glycoprotein of human immunodeficiency virus type 1. J Virol 70:1100-8.
61. Buchacher A, Predl R, Strutzenberger K, Steinfellner W, Trkola A, Purtscher M, Gruber G, Tauer C, Steindl F, Jungbauer A, et al. 1994. Generation of human monoclonal antibodies against HIV-1 proteins: electrofusion and Epstein-Barr virus transformation for peripheral blood lymphocyte immortalization. AIDS Res Hum Retroviruses 10:359-69.
62. Mouquet H, Scharf L, Euler Z, Liu Y, Eden C, Scheid J F, Halper-Stromberg A, Gnanapragasam P N, Spencer D I, Seaman M S, Schuitemaker H, Feizi T, Nussenzweig M C, Bjorkman P J. 2012. Complex-type N-glycan recognition by potent broadly neutralizing HIV antibodies. Proc Natl Acad Sci USA 109: E3268-77.
63. Gorny M K, Wang X -H, Williams C, Volsky B, Revesz K, Witover B, Burda S, Urbanski M, Nyambi P, Krachmarov C, Pinter A, Zolla-Pazner S, Nadas A. 2009. Preferential use of the VH5-51 gene segment by the human immune response to code for antibodies against the V3 domain of HIV-1. Molecular Immunology 46:917-926.
64. Gomy M K, Conley A J, Karwowska S, Buchbinder A, Xu J Y, Emini E A, Koenig S, Zolla-Pazner S. 1992. Neutralization of diverse human immunodeficiency virus type 1 variants by an anti-V3 human monoclonal antibody. J Virol 66:7538-42.
65. Huang J, Kang B H, Pancera M, Lee J H, Tong T, Feng Y, Imamichi H, Georgiev I S, Chuang G Y, Druz A, Doria-Rose N A, Laub L, Sliepen K, van Gils M J, de la Peña A T, Derking R, Klasse P J, Migueles S A, Bailer RT, Alam M, Pugach P, Haynes B F, Wyatt R T, Sanders R W, Binley J M, Ward A B, Mascola JR, Kwong P D, Connors M. 2014. Broad and potent HIV-1 neutralization by a human antibody that binds the gp41-gp120 interface. Nature 515:138-42.
66. Zolla-Pazner S, Alvarez R, Kong X -P, Weiss S. 2019. Vaccine-induced VIV2-specific antibodies control and or protect against infection with HIV, SIV and SHIV. Current Opinion in HIV and AIDS 14.
67. Sanders R W, Moore J P. 2017. Native-like Env trimers as a platform for HIV-1 vaccine design. Immunological reviews 275:161-182.
68. Bontempo A, Garcia M M, Rivera N, Cayabyab M J. 2020. A Systematic Approach to HIV-1 Vaccine Immunogen Selection. AIDS Res Hum Retroviruses 36:762-770.
69. Mayr L M, Cohen S, Spurrier B, Kong X -P, Zolla-Pazner S. 2013. Epitope mapping of conformational V2-specific anti-HIV human monoclonal antibodies reveals an immunodominant site in V2. PloS one 8:e70859-e70859.
70. Sok D, Burton D R. 2018. Recent progress in broadly neutralizing antibodies to HIV. Nat Immunol 19:1179-1188.
71. Ringe R P, Sanders R W, Yasmeen A, Kim H J, Lee J H, Cupo A, Korzun J, Derking R, van Montfort T, Julien J P, Wilson I A, Klasse PJ, Ward AB, Moore J P. 2013. Cleavage strongly influences whether soluble HIV-1 envelope glycoprotein trimers adopt a native-like conformation. Proc Natl Acad Sci USA 110:18256-61.
72. Yasmeen A, Ringe R, Derking R, Cupo A, Julien J P, Burton D R, Ward A B, Wilson I A, Sanders R W, Moore J P, Klasse P J. 2014. Differential binding of neutralizing and non-neutralizing antibodies to native-like soluble HIV-1 Env trimers, uncleaved Env proteins, and monomeric subunits. Retrovirology 11:41.
73. Nyambi P N, Mbah H A, Burda S, Williams C, Gorny M K, Nádas A, Zolla-Pazner S. 2000. Conserved and exposed epitopes on intact, native, primary human immunodeficiency virus type 1 virions of group M. J Virol 74:7096-107.
74. van Eeden C, Wibmer C K, Scheepers C, Richardson S I, Nonyane M, Lambson B, Mkhize N N, Vijayakumar B, Sheng Z, Stanfield-Oakley S, Bhiman J N, Bekker V, Hermanus T, Mabvakure B, Ismail A, Moody M A, Wiehe K, Garrett N, Karim S A, Dirr H, Fernandes M A, Sayed Y, Shapiro L, Ferrari G, Haynes B F, Moore PL, Morris L. 2018. V2-Directed Vaccine-like Antibodies from HIV-1 Infection Identify an Additional K169-Binding Light Chain Motif with Broad ADCC Activity. Cell Rep 25:3123-3135.e6.
75. Mclellan J S, Pancera M, Carrico C, Gorman J, Julien J -P, Khayat R, Louder R, Pejchal R, Sastry M, Dai K, O'Dell S, Patel N, Shahzad-ul-Hussan S, Yang Y, Zhang B, Zhou T, Zhu J, Boyington J C, Chuang G -Y, Diwanji D, Georgiev I, Kwon Y D, Lee D, Louder M K, Moquin S, Schmidt S D, Yang Z -Y, Bonsignori M, Crump J A, Kapiga S H, Sam N E, Haynes B F, Burton D R, Koff W C, Walker L M, Phogat S, Wyatt R, Orwenyo J, Wang L-X, Arthos J, Bewley C A, Mascola J R, Nabel G J, Schief W R, Ward A B, Wilson I A, Kwong P D. 2011. Structure of HIV-1 gp120 V1/V2 domain with broadly neutralizing antibody PG9. Nature. 480(7377): 336-343. doi: 10.1038/nature10696.

### ADDITIONAL REFERENCES

Rao, V. R. et al. Clade C HIV-1 isolates circulating in Southern Africa exhibit a greater frequency of dicysteine motif-containing Tat variants than those in Southeast Asia and cause increased neurovirulence. Retrovirology 10, (2013). http://www.retrovirology.com/content/10/1/61
Ahmed, S. et al. Stabilization of a soluble, native-like trimeric form of an efficiently cleaved Indian HIV-1 clade C envelope glycoprotein. J. Biol. Chem. 292, 8236-8243 (2017).
NIAID. First New HIV Vaccine Efficacy Study in Seven Years Has Begun. https://www.niaid.nih.gov/news-events/first-new-hiv-vaccine-efficacy-study-seven-years-has-begun (2016).
Evaluating the Safety and Immunogenicity of ALVAC-HIV and MF59®- or AS01B-adjuvanted Bivalent Subtype C gp120 in Healthy, HIV-uninfected Adult Participants (HVTN 120). https://clinicaltrials.gov/ct2/show/NCT03122223 (2017).
U.S. Patent Application Publication 2016/0272948 A1.
Schorcht, A. et al. Neutralizing Antibody Responses Induced by HIV-1 Envelope Glycoprotein SOSIP Trimers Derived from Elite Neutralizers. Journal of Virology 94(24): 1-25 (2020). https://doi.org/10.1128/JV1.01214-20
Volz A, Sutter G. Modified Vaccinia Virus Ankara: History, Value in Basic Research, and Current Perspectives for Vaccine Development. Adv Virus Res. 2017;97:187-243. doi: 10.1016/bs.aivir.2016.07.001. Epub 2016 Aug. 1. PMID: 28057259; PMCID: PMC7112317.
Capucci S, Wee E G, Schiffner T, LaBranche C C, Borthwick N, Cupo A, et al. (2017) HIV-1-neutralizing antibody induced by simian adenovirus- and poxvirus MVA-vectored BG505 native-like envelope trimers. PLOS ONE 12(8): e0181886. https://doi.org/10.1371/journal.pone.0181886
Sahoo. S. et al. Structure-guided changes at the V2 apex of HIV-1 clade C trimer enhance elicitation of autologous neutralizing and broad VIV2-scaffold antibodies. Cell Reports. 38:110436 (2022). https://doi.org/10.1016/j.celrep.2022.110436

1-31. (canceled)

32. A trimer, wherein the trimer is a non-naturally occurring clade C human immunodeficiency virus type-1 (HIV-1) 1086.c envelope (ENV) trimer having a form stabilized with a disulfide SOSIP mutation and having a mutation that replaces an isoleucine residue at position 559 with a proline residue.

33. The trimer according to claim 32, wherein the trimer contains at least one of a broadly neutralizing epitope and an epitope that induces anti-V1/V2 antibodies.

34. The trimer according to claim 32, wherein a nucleic acid encoding the trimer has a sequence of SEQ ID NO:1.

35. A pharmaceutical composition comprising the trimer according to claim 32 and at least one of an adjuvant and a pharmaceutically acceptable carrier.

36. The pharmaceutical composition according to claim 35, wherein the trimer contains at least one of a broadly neutralizing epitope and an epitope that induces anti-V1/V2 antibodies.

37. The pharmaceutical composition according to claim 35, further comprising a different HIV-1 envelope (ENV) trimer from clade C or from a clade type other than clade C.

38. The pharmaceutical composition according to claim 37, wherein the different HIV-1 envelope (ENV) trimer is from a clade type other than clade C and is a clade A BG505 SOSIP trimer.

39. The pharmaceutical composition according to claim 35, wherein the trimer is expressed by DNA, RNA, mRNA, or by a recombinant viral vector.

40. The pharmaceutical composition according to claim 39, wherein the trimer is expressed by a recombinant viral vector including adenovirus or Modified Vaccinia Virus Ankara (MVA).

41. An immunogenic composition for eliciting an immune reaction in cells against clade C human immunodeficiency virus type-1 (HIV-1), the immunogenic composition comprising:
a non-naturally occurring clade C human immunodeficiency virus type-1 (HIV-1) 1086.c envelope (ENV) trimer having a form stabilized with a disulfide SOSIP mutation and having a mutation that replaces an isoleucine residue at position 559 with a proline residue; and
at least one of an adjuvant and a pharmaceutically acceptable carrier.

42. The immunogenic composition according to claim 41, wherein a nucleic encoding the non-naturally occurring clade C human immunodeficiency virus type-1 (HIV-1) 1086.c envelope (ENV) trimer has a sequence of SEQ ID NO:1.

43. The immunogenic composition according to claim 41, further comprising a different HIV-1 envelope (ENV) trimer from clade C or from a clade type other than clade C.

44. The immunogenic composition according to claim 43, wherein the different HIV-1 envelope (ENV) trimer is from a clade type other than clade C and is a clade A BG505 SOSIP trimer.

45. A prophylactic or therapeutic vaccine against HIV-1 comprising the trimer according to claim 32 and at least one of a pharmaceutically acceptable carrier, an adjuvant, and a different HIV-1 envelope (ENV) trimer from clade C or from a clade type other than clade C.

46. A kit comprising the prophylactic or therapeutic vaccine against HIV-1 according to claim 45 or individual elements of the prophylactic or therapeutic vaccine against HIV-1 according to claim 45.

47. A method for inducing an immune response against clade C human immunodeficiency virus type-1 (HIV-1) infection in a subject in need thereof, the method comprising:
providing a composition comprising a non-naturally occurring clade C human immunodeficiency virus type-1 (HIV-1) 1086.C envelope (ENV) trimer having a form stabilized with a disulfide SOSIP mutation and having a mutation that replaces an isoleucine residue at position 559 with a proline residue, and at least one of a pharmaceutically acceptable carrier, an adjuvant, and an HIV-1 envelope (ENV) trimer from a clade type other than clade C; and
administering the composition to the subject.

48. A method for eliciting an immune reaction in cells against clade C human immunodeficiency virus type-1 (HIV1) infection, the method comprising:
providing the pharmaceutical composition according to claim 35; and
administering the pharmaceutical composition to the cells.

49. A method for inducing production of broadly neutralizing antibodies (bnAbs) against clade C human immunodeficiency virus type-1 (HIV-1) infection in a subject in need thereof, the method comprising:
providing the pharmaceutical composition according to claim 35; and
administering the pharmaceutical composition to the subject.

50. A method for inducing production of antibodies against human immunodeficiency virus type-1 (HIV-1) V2 apex in a subject in need thereof, the method comprising:
providing the pharmaceutical composition according to claim 35; and
administering the pharmaceutical composition to the subject.

51. A method for vaccinating a subject against clade C human immunodeficiency virus type-1 (HIV1) infection, the method comprising:
providing the pharmaceutical composition according to claim 35; and
administering the pharmaceutical composition to the subject.
